(19) United States
(12) Patent Application Publication
(10) Pub. No.: US 2026/0340508 A1
(43) Pub. Date: Oct. 29, 2026

(54) COMPUTER-IMPLEMENTED METHOD FOR ANALYZING EXPERIMENTAL DATA AND COMPUTER-IMPLEMENTED METHOD OF GENERATING TRAINING DATA FOR TRAINING A MACHINE LEARNING MODEL FOR ANALYZING EXPERIMENTAL DATA

(71) Applicants: Max-Planck-Gesellschaft zur Förderung der Wissenschaften e.V., Munich (DE); Ludwig-Maximilians-Universität München, in Vertretung des Freistaates Bayern, Munich (DE)

(72) Inventors: Tarek Abdelmaksoud Abdalla Eissa, Munich (DE); Marinus Huber, Holzkirchen (DE); Mihaela Zigman Kohlmaier, Munich (DE)

(73) Assignees: Max-Planck-Gesellschaft zur Förderung der Wissenschaften e.V., Munich (DE); Ludwig-Maximilians-Universität München, in Vertretung des Freistaates Bayern, Munich (DE)

(21) Appl. No.: 19/713,794

(22) Filed: Jun. 19, 2026

Related U.S. Application Data
(63) Continuation of application No. PCT/EP2024/065485, filed on Jun. 5, 2024.

(30) Foreign Application Priority Data
Dec. 20, 2023 (EP) 23218875.5

(51) Int. Cl. G06N 20/00

(52) U.S. Cl. CPC G06N 20/00

(57) ABSTRACT

A computer-implemented method analyzes experimental data. The method comprises receiving a first data set including the experimental data to be analyzed. The method further comprises receiving one or more variation data sets, wherein each variation data set comprises information regarding a variation of experimental observations. Moreover, the method comprises extrapolating the experimental data by applying a variation to the experimental data, wherein the applied variation corresponds to the one or more variations of the experimental observations specified by the information comprised in the one or more variation data sets. The method further comprises generating a synthetic data set based on the extrapolated experimental data, analyzing the synthetic data set, and providing an output based on a result of the analysis of the synthetic data set. The received one or more variation data sets are selected depending on types of noise related to the experimental data to be analyzed.

### CROSS REFERENCE TO RELATED APPLICATION

This application is a continuation application of international patent application PCT/EP2024/065485, filed on Jun. 5, 2024, and designating the U.S., which claims priority to European patent application 23218875.5 filed on Dec. 20, 2023, each of which is hereby incorporated by reference in their entireties.

### TECHNICAL FIELD

Provided is a computer-implemented method for analyzing experimental data, a computer-implemented method of generating training data for training a machine learning model for analyzing experimental data, a Computer-implemented method for training a machine learning model for analyzing experimental data, a Data processing device, a Computer-implemented machine learning model for analyzing experimental data, a use of the computer-implemented machine learning model according to the disclosure for analyzing experimental data, a Computer-implemented method for classifying experimental data being subject to variation using a machine learning model, and a method of identifying an individual based on a specimen of a biofluid of the individual. The disclosure is, thus, related to the analysis of experimental data by using a machine learning model and/or assisted by a machine learning model.

### BACKGROUND

Technological advances in molecular analytics increasingly enable the probing of biological systems. Distinguishing between physiologically relevant states from quantitative molecular fingerprints presents a new opportunity for in vitro phenotyping. Extensive efforts are thus dedicated to developing standardized procedures involving streamlined biological sampling, post-collection handling, and sensitive quantitative measurements. Nevertheless, empirical datasets remain susceptible to diverse sources of variability, both analytical and inherently biological (see R. A. Bowen and A. T Remaley, “Interferences from blood collection tube components on clinical chemistry assays,” Biochemia Medica, pp. 31-44, 2014). Obtaining a dataset that reflects a realistic data distribution is often resource-intensive, costly, and, in some cases, virtually impossible. This is especially true in the context of clinical studies, covering all pathophysiological strata, studying rare disease, or longitudinally probing the same system over time. Exploratory studies are thus often limited in sample size, making it challenging for a given “training” set to be representative of the true unseen “test” domain. Consequently, when applying a developed machine learning (ML) model to independently collected and experimentally measured samples, the model may fall short of achieving the expected efficacy.

While traditional approaches often rely on standardizing experimental workflows and creating computer-aided processing techniques to reduce unwanted empirical noise, complete noise removal seems likely unattainable (see C. L. M. Morais, K. M. G. Lima, M. Singh, and F. L. Martin, “Tutorial: multivariate classification for vibrational spectroscopy in biological samples,” Nature Protocols, vol. 15, pp. 2143-2162, June 2020). Failure to account for noise and distributional shifts may mask the true biological patterns of interest, potentially misleading an ML algorithm to utilize confounding information that is unlikely to be reproduced. This failure is due to violating the assumption underlying (supervised) ML algorithms that the training and testing data are independent and identically distributed (i.i.d.) (see J. G. Moreno-Torres, T Raeder, R. Alaiz-Rodriguez, N. V Chawla, and F. Herrera, “A unifying view on dataset shift in classification,” Pattern Recognition, vol. 45, pp. 521-530, January 2012). To decode the information that a collected dataset may hold, understanding and accounting for analytical and biological variability may be critical to ensure successful data analysis. The concept of out-of-distribution (OOD) generalization has very recently garnered attention in ML research to address the shortcomings of i.i.d. assumptions (see J. Liu, Z. Shen, Y. He, X Zhang, R. Xu, H. Yu, and P. Cui, “Towards out-of-distribution generalization: A survey”, arXiv, 2023). This paradigm shift acknowledges the unpredictability of unseen data, prompting exploration of methods that better accommodate distributional shifts to generalize beyond the training set. OOD generalization has been extensively explored in computer vision and natural language processing tasks (see X Zhang, L. Zhou, R. Xu, P. Cui, Z. Shen, and H. Liu, “Towards unsupervised domain generalization,” arXiv, 2022). However, there is a critical lack in the development of OOD generalization techniques, in particular in molecular analytics involving vibrational spectroscopy, NMR spectroscopy, and mass spectrometry, as well as in clinical chemistry analytics.

The following publication describes a method of data augmentation of spectral data for convolutional neural network based deep chemometrics, according to which some of the datasets were augmented by adding random variations in offset, multiplication and slope: E. J. Bjerrum et al., Data Augmentation of Spectral Data for Convolutional Neural Network (CNN) Based Deep Chemometrics, arXiv, 2017.

### SUMMARY

It is the objective technical problem of the disclosure to enrich the prior art. Optionally, the objective technical problem may be permitting an analysis of experimental data with improved reliability even when the experimental data comprises only a limited number of data points.

The objective technical problem is solved by the subject-matter of the independent claims. Optional features and embodiments are presented in the dependent claims and in the description.

Provided is a computer-implemented method for analyzing experimental data. The method comprises receiving a first data set including the experimental data to be analyzed, and receiving one or more variation data sets, wherein each variation data set comprises information regarding a variation of experimental observations. The method further comprises extrapolating the experimental data by applying a variation to the experimental data, wherein the applied variation corresponds to the one or more variations of the experimental observations specified by the information comprised in the one or more variation data sets. Moreover, the method comprises generating a synthetic data set based on the extrapolated experimental data, analyzing the synthetic data set, and providing an output based on a result of the analysis of the synthetic data set. The received one or more variation data sets that are received are selected depending on one or more types of noise related to the experimental data to be analyzed.

Furthermore, a computer program is provided, the computer program comprising instructions which, when the program is executed by a computer, cause the computer to carry out a method according to the disclosure.

Moreover, a computer-readable medium is provided, optionally a computer-readable storage medium and/or a data carrier signal, comprising instructions which, when executed by a computer, cause the computer to carry out the method according to the disclosure.

In addition, a data processing device configured to carry out the method according to the disclosure is provided.

In yet another aspect, a computer-implemented method of generating training data for training a machine learning model for analyzing experimental data is provided. The method comprises receiving one or more variation data sets, wherein each variation data set comprises experimental observations being subject to a variation at least partially affecting the experimental data to be analyzed. The method further comprises analyzing the variation of the experimental observations comprised in the one or more variation data sets and determining variability properties based on the analyzed variation. Moreover, the method comprises receiving a first data set including at least one reference specimen of the experimental data and a predetermined analysis result for the at least one reference specimen; and generating one or more synthetic data sets including multiple synthetic data points by applying the determined variability properties to the first data set.

Furthermore, a computer-implemented method for training a machine learning model for analyzing experimental data is provided. The method comprises receiving a training data set comprising training data, wherein the training data comprises one or more synthetic data sets having the same or a similar variation as one or more variation data sets used for generating the one or more synthetic data sets based on a first data set including at least one reference specimen of the experimental data and a predetermined analysis result for the at least one reference specimen. The method further comprises training the machine learning model with the received training data set.

Moreover, a computer-implemented method for analyzing experimental data by using a machine learning model is provided, wherein the machine learning model is configured to classify the analyzed experimental data being subject to a variation exceeding a variation of reference specimen of the experimental data used as a first data set for generating the training data for training the machine learning model.

In addition, a computer implemented method of analyzing experimental data by using a machine learning model, wherein the machine learning model is trained by a method according to the disclosure.

Furthermore, a computer program is provided, the computer program comprising instructions which, when the program is executed by a computer, cause the computer to carry out a method according to the disclosure.

In addition, a computer-readable medium, optionally a computer-readable storage medium and/or a data carrier signal is provided, the computer-readable medium comprising instructions which, when executed by a computer, cause the computer to carry out the method according to the disclosure.

Moreover, a data processing device configured to carry out the method according to the disclosure is provided.

In addition, a computer-implemented machine learning model for analyzing experimental data is provided, wherein the machine learning model is configured to analyze experimental data being subject to a variation exceeding a variation of reference specimen of the experimental data used as a first data set for generating the training data for training the machine learning model.

Yet, a use of the computer-implemented machine learning model according to the disclosure for analyzing experimental data is provided.

Furthermore, a computer-implemented method for classifying experimental data being subject to variation using a machine learning model is provided, wherein the variation of the experimental data to be classified exceeds a variation of a reference specimen of the experimental data used for generating the training data for training the machine learning model.

In addition, a method of identifying an individual based on a specimen of a biofluid of the individual is provided. The method comprises receiving experimental data characterizing the specimen of the biofluid of the individual. The method further comprises determining an analysis result for the received experimental data by using a machine learning model according to the disclosure trained on experimental data originating in a reference specimen of a biofluid of the individual, wherein the analysis result corresponds to a match or a mismatch between the received experimental data characterizing the specimen and the experimental data of the reference specimen used for training the machine learning model. Furthermore, the method comprises providing an information regarding the identification of the individual based on the match or mismatch between the received experimental data characterizing the specimen and the experimental data of the reference specimen used for training the machine learning model.

Moreover, a computer-implemented method of generating data for analyzing experimental data is provided. The method comprises receiving one or more variation data sets, wherein each variation data set comprises experimental observations being subject to a variation at least partially affecting the experimental data to be analyzed. Furthermore, the method comprises analyzing the variation of the experimental observations comprised in the one or more variation data sets and determining variability properties based on the analyzed variation. In addition, the method comprises receiving a first data set including at least one reference specimen of the experimental data and a predetermined analysis result for the at least one reference specimen, and generating one or more synthetic data sets including multiple synthetic data points by applying the determined variability properties to the first data set.

“Analyzing experimental data” may relate to extracting information from data, wherein the data was or is retrieved in an experimental process. The experimental process may include scientific experiments. The experimental data may be subject to various measurement errors and/or other sources of noise and variation originating from the measurements and/or the process of generating the specimen examined in the experimental process and/or the nature of the specimen itself.

A variation data set is a set of data comprising information regarding a variation of experimental observations. A variation of experimental observations is to be understood as noise to which the experimental observations are subject. The variation data set may comprise a plurality of data values representing a variation of a particular data point of the experimental observation. The variation data may represent a “point cloud”, which is to be understood as a “cloud” of data points spread out by noise, which are related to a particular data point representing at least a part of the experimental observation. The variation data set may comprise the point cloud itself. Alternatively or additionally, the variation data set may comprise a mathematical description of a variation to which an underlying experimental observation is subject. An experimental observation may be or comprise a result of an experimental measurement and/or experimental data of any other origin. The experimental observation may be spectroscopic measurement data comprising spectral information about a specimen from which the spectroscopic information was retrieved.

“Extrapolating the experimental data” may refer to adding data points and/or other data to the experimental data, which originally was not part of the experimental data. Extrapolating the experimental data may comprise adding one or more point clouds to one or more data points included in the experimental data, wherein the one or more point clouds are defined by the information comprised in the one or more variation data sets.

“Generating a synthetic data set” may be understood as creating a data set based on the first data set and the one or more variation data sets, wherein the extrapolated data are used to create the synthetic data set. The term “synthetic” meaning that the synthetic data set is generated in an artificial manner involving the extrapolated experimental data and does not solely include measured experimental data.

“Analyzing the synthetic data set” is to be understood as analyzing the experimental data such as to extract at least the information from the synthetic data set that is aimed for by analyzing the experimental data. Analyzing the synthetic data may comprise the same steps that are conventionally carried out based on the (original) experimental data with the difference that the synthetic data set is analyzed. Optionally, the process of analyzing the synthetic data may differ from a conventional process of analyzing the experimental data in the process steps carried out. In other words, in addition to replacing the experimental data with the synthetic data set, the analysis process may comprise additional modifications with respect to the conventional process of analyzing the experimental data.

The one or more variation data sets being selected depending on one or more types of noise related to the experimental data to be analyzed is to be understood such that the one or more received variation data sets are or have been chosen in consideration of the one or more types of noise related to the experimental data to be analyzed. In other words, the one or more variation data sets may be chosen in a contextual manner such that a context or connection between the variation data set and the experimental data to be analyzed is considered. The one or more received variation data sets may depend in such a manner on the one or more types of noise related to the experimental data that the variations of experimental observations being subject to the information comprised by the one or more originate in the same types of noise which are expected or known to typically affect the experimental data to be analyzed.

A machine learning model may be understood as an algorithm based on artificial intelligence, which may be understood as an algorithm that defines an artificial system that has learned correlations from examples or based on training data as part of a learning phase and can generalize these after the end of the learning phase (so-called machine learning). The algorithm can have a statistical model that is based on training data. This means that the machine learning described above does not memorize the examples from the training data, but rather recognizes patterns and regularities in the learning or training data. In this way, the algorithm can also evaluate unknown data outside of the training data set (so-called learning transfer or generalization).

The machine learning model can have at least one artificial neural network. An artificial neural network can be based on several interconnected units or nodes, which are referred to as artificial neurons. A connection between the neurons may transmit information (sometimes referred to as a signal) to one or more other neurons. An artificial neuron receives input information, processes the input information and can then output information based on the processing of the input information to the neuron or neurons connected to it. The input and output information can be a real number, whereby the output information of the respective neuron can be calculated by a function, in particular a non-linear function, as the sum of its input information. The connections can also be referred to as edges. Neurons and edges may have a weighting, which is adjusted during the learning process or a training procedure by means of a loss function to be optimized. The weighting increases or decreases the output information. Neurons can have a threshold value so that output information is only sent if the output information exceeds this threshold value. Neurons can be grouped in layers. Different layers can perform different transformations on their inputs. The information may be sent from a first layer (the input layer) to a last layer (the output layer), possibly via intermediate layers, possibly after having passed through one or more of the layers several times. More specifically, the neurons may be arranged in multiple layers, where neurons of one layer, optionally only, may be connected to neurons of the immediately preceding and immediately following layers. The layer that receives external data is the input layer. The layer that produces the final result is the output layer. There can be zero or more hidden layers in between. A single-layer network can also be used. Several connection patterns are possible between two layers. The two layers can be fully connected, i.e. every neuron in one layer can be connected to every neuron in the next layer. However, the layers can also be connected by “pooling”, i.e. a group of neurons in one layer connects to a single neuron in the next layer, thereby reducing the number of neurons in the next layer. Networks that only have such connections between the layers form a directed acyclic graph and are called feedforward networks. Alternatively, networks that allow connections between neurons in the same or previous layers are called recurrent networks.

The synthetic data set is, however, not limited to machine learning applications. The synthetic data set generated may be used for purposes of basic statistical analysis—e.g., calculating a mean, standard deviation, and range of values for some experimental data after introducing additional levels of variability to them. The synthetic data set data generated may be applied to perform more accurate outlier detection, data clustering, and/or data visualization.

The disclosure provides the advantage that a reliability of an analysis of experimental data can be improved. When analyzing experimental data having only a very limited number of samples or even only one sample, the disclosure may increase the reliability of the analysis process when carried out based on the synthetic data set as compared to the first data set.

Moreover, the disclosure provides the advantage that the experimental data can be enriched by a realistic distribution of data points which is expected for the type of the experimental data but is not exhibited by the experimental data due to a small number of samples available.

The disclosure provides the advantage that the analysis of experimental data by using a machine learning model may be facilitated. In particular, the disclosure may provide the advantage that an accuracy and/or a reliability of an analysis of the experimental data, such as a classification of the experimental data, may be increased despite only a small number of reference specimens of the experimental data are used for training the machine learning model.

The disclosure may provide the advantage that a machine learning model may be enabled to analyze experimental data at a high accuracy and/or high reliability although the analyzed data may be subject to a higher level of variation than a possibly rather limited first data set used for training the machine learning model.

The disclosure may further provide the advantage that knowledge about variations possibly affecting the experimental data to be analyzed may be considered and applied in the process of training the machine learning model. This may allow enabling the machine learning model to cope with experimental data having a higher degree of variation than the reference specimen of the experimental data available for training the machine learning model. Therefore, the disclosure may provide the advantage that the machine learning model may be robust and enabled for out-of-distribution generalization.

The one or more variation data sets may be selected such that the variation of experimental observations contained in the one or more data sets correspond to noise typically affecting experimental data of the same type as the experimental data included in the first data set. In other words, the selected noise may be selected to match the noise typically affecting experimental data to be analyzed, which may be, however, absent due to a small number of available samples. This may increase the accuracy for the analysis of the experimental data.

The received one or more variation data sets may be selected in a contextual manner based on a context related to the experimental data to be analyzed and/or based on a context related to the analysis of the experimental data to be performed. The context may arise from the type of experimental data and/or the preparation of the samples for retrieving the experimental data and/or the measurements for retrieving the experimental data. Different processes may induce different kinds of noise. The context dependency may consider in particular such variation data sets which reflect the types of noise which are expected to affect or have affected the samples and/or the process for retrieving the experimental data.

The received one or more variation data sets are at least partially selected and/or provided by an external source. Optionally, the selection may be carried out by a user and may be provided by means of a user input. Optionally an external computation device, such as a remote server, may be configured to select the variation data sets to be received for analyzing the experimental data. The selection of the variation data sets to be received may involve examining the experimental data to be analyzed. This may provide the advantage that it allows for an effective selection of the variation data sets to be received.

The method may further comprise, prior to receiving the one or more variation data sets, a step of determining one or more types of noise to be considered when analyzing the measurement data, and a step of selecting the one or more variation data sets to be received based on the determined one or more types of noise. In other words, the selection of the one or more variation data sets may be at least partially constitute part of the method for analyzing the experimental data. In other words, the selection of the one or more variation data sets may be at least partially carried out by a data processing device carrying out the method for analyzing the experimental data. The method may further comprise requesting the selected one or more variation data sets from an external data source. This may provide the advantage that an adaptable selection of the one or more variation data sets may be integrated in the process of analyzing the experimental data by a data processing device carrying out the method for analyzing the experimental data.

The experimental observations of the one or more variation data sets may be independent of the experimental data included in the received first data set. In other words, the experimental observations of the one or more variation data sets may have been retrieved entirely independently of the retrieval of the experimental data to be analyzed. The experimental observations of the one or more variation data sets may have been retrieved from samples being independent of the samples from which the experimental data to be analyzed have been retrieved. The samples for retrieving the experimental observations of the one or more variation data sets may be samples which allow for a retrieval of a distribution of data points originating in a particular kind of noise. Optionally, the samples for retrieving the experimental observations of the one or more variation data sets may be samples which allow for a retrieval of a distribution of data points originating in a particular kind of noise with only little or no interference of other kinds of noise other than the particular kind of noise. The retrieved data points may then be used as experimental observations for one or more variation data sets specifying said particular kind of noise. These one or more variation data sets may then be selected or received when said particular kind of noise shall be considered in the analysis of the experimental data to be analyzed.

Extrapolating the experimental data may comprise or consist of adding a measurement point cloud for at least one measurement point of the experimental data, wherein the measurement point cloud is based on at least one of the one or more variation data sets. Different types of noise and/or different variation data sets may affect different parts of the experimental data, such as optionally different spectral features of spectral data to be analyzed. The extrapolation may comprise adding a statistical distribution of data points to one or more particular data points comprised in the experimental data.

The one or more variation data sets may be selected such that the experimental observations of the one or more variation data sets and the experimental data included in the first data set are of the same type of experimental data. The selection of variation data set may be adapted to select variation data sets reflecting such types of noise which were retrieved from samples being of the same type of the samples from which the experimental data to be analyzed has been retrieved. This may allow adding an accurate statistical distribution of data points when extrapolating the experimental data.

The type of the experimental data may relate to spectroscopic data. The experimental data may include data retrieved by at least one of the following techniques:
vibrational spectroscopy;
optical spectroscopy in the visible and/or ultraviolet spectral range;
X-ray diffraction;
NMR spectroscopy;
mass spectrometry;
clinical chemistry tests;
electrophysiology; and
flow cytometry.

Different types of noise may affect different spectral features of the spectral data forming part of the experimental data. Accordingly, different variation data sets may result in variations of different spectral features of the experimental data. However, some types of noise and accordingly some variation data sets may affect all spectral features of the experimental data to be analyzed.

The experimental data may be retrieved from a biofluid of a biological organism. The biofluid may include at least one of the following types:
blood;
plasma;
serum;
saliva;
urine or exprimate urine;
cerebrospinal fluid;
living or chemically fixed animal, human, plant tissue;
living or chemically fixed bacterial, animal, human, plant cells;
viruses or viral particles; and
multimolecular structures.

The variation of the experimental observations contained in a particular variation data set of the one or more variation data sets may originate in one of the following sources of variation:
a tolerance in quantitative measurement reproducibility of a measurement technique used for retrieving the experimental observations;
a preanalytical variability occurring in a process for preparing one or more samples for retrieving the experimental observations;
a variation between different phenotypes from which the one or more samples for retrieving the experimental observations originate;
a variation that is inherent to the studied biological system, encompassing both variations between different individuals and intra-individual variations when studied over time.

The variations may arise from differences in sample storage, such as storage durations and/or storage conditions. The variations may arise from differences in sample handling, such as handling by different machines and/or human operators and/or handling durations and/or handling conditions. The variations may arise from differences in the retrieval of the experimental data from the samples, such as differences in the apparatuses used for carrying out the measurements and/or settings of the apparatuses for carrying out the measurements and/or varying performances of the apparatuses used for carrying out the measurements.

The number of synthetic data points may be larger than the number of the at least one reference specimen of the experimental data included in the first data set. This may allow providing a synthetic data set being more extensive than the first data set for training a machine learning model than the use of the first data set would allow when used for training a machine learning model.

The received one or more variation data sets may be selected, such that the variation of the experimental observations comprised in the one or more variation data sets corresponds to an actual variation and/or an expected variation affecting the experimental data to be analyzed. This may provide the advantage that the training data may be adapted to the variation affecting the experimental data to be analyzed.

The variation affecting the experimental data to be analyzed may originate in one or more of the following variation sources: (i) a tolerance in quantitative measurement reproducibility of a measurement technique used for retrieving the experimental data, (ii) a preanalytical variability occurring in a process for preparing one or more samples for retrieving the experimental data, (iii) a variation between different phenotypes from which the one or more samples for retrieving the experimental data originate; and (iv) a variation between types of samples from which the experimental data is extracted; and (v) a biological variation between different biological systems or the same biological system over time.

Each variation data set may comprise experimental observations affected by a variation originating in one or more predetermined variation sources. Alternatively or additionally, each variation data set optionally comprises experimental observations affected solely by a variation originating in one or more predetermined sources of the variation sources.

Each variation data set may comprises experimental observations affected by a variation originating in one or more predetermined variation sources, wherein the experimental observations are essentially unaffected by variations originating in variation sources different from the predetermined variation sources.

The experimental data may include data retrieved by at least one of the following techniques: (i) vibrational spectroscopy, (ii) optical spectroscopy in the infrared, visible and/or ultraviolet spectral range, (iii) X-ray diffraction, (iv) NMR spectroscopy, (v) mass spectrometry, (vi) clinical chemistry tests, (vii) electrophysiology and (viii) flow cytometry.

The experimental data may be retrieved from a biofluid of a biological organism. The biofluid may include at least one of the following types: (i) blood, (ii) plasma, (iii) serum, (iv) saliva, (v) urine or exprimate urine, (vi) cerebrospinal fluid, (vii) living of chemically fixed animal, human, plant tissue, (viii) living of chemically fixed bacterial, animal, human, plant cells, (ix) viruses or viral particles, (x) multimolecular structures. Multimolecular structures may include at least one of (i) amyloids, (ii) antibodies, (iii) antigen, and (iv) other molecular complexes.

The reference specimen of the experimental data included in the first data set may be retrieved from the same biofluid type as the experimental data to be analyzed by the machine learning model to be trained with the one or more synthetic data sets. Alternatively, the reference specimen of the experimental data included in the first data set may be retrieved from a different type biofluid as the experimental data to be analyzed by the machine learning model to be trained with the one or more synthetic data sets. The machine learning model may be trained with training data having a variation reflecting these possible variations in the experimental data.

The one or more variation data sets may be selected such that the experimental observations comprised in the one or more variation data sets are affected by the same and/or a similar type of variation as the experimental data to be analyzed.

The variation of the experimental observations comprised in a particular variation data set of the one or more variation data sets may originate in one of the following sources of variation: (i) a tolerance in quantitative measurement reproducibility of a measurement technique used for retrieving the experimental observations, (ii) a preanalytical variability occurring in a process for preparing one or more samples for retrieving the experimental observations, and (iii) a variation between different phenotypes from which the one or more samples for retrieving the experimental observations originate.

Analyzing the variation of the experimental observations comprised in the one or more variation data sets may include analyzing the variation of the experimental observations of each of the one or more variation data sets independently of each other. Alternatively or additionally, determining the variability properties based on the analyzed variation of the experimental observations may include determining the variability properties based on the experimental observations for each of the one or more variation data sets independently of each other.

Applying the determined variability properties to the first data set may comprise extrapolating each of at least some of the one or more data points of the reference specimen of the experimental data included in the first data to multiple synthetic data points according to the variability properties.

The synthetic data set may be provided as training data for training a machine learning model for analyzing experimental data based on out-of-distribution generalization.

The variation of the experimental data to be classified may correspond to a variation of one or more synthetic data sets used for training the machine learning model, wherein the one or more synthetic data sets are based on a first data set including the at least one reference specimen of the experimental data, a predetermined analysis result for the at least one reference specimen, and on one or more variation data sets comprising experimental observations being subject to a variation at least partially affecting the experimental data to be classified. The machine learning model may be a machine learning model according to the disclosure.

The disclosure provided for the method for analyzing experimental data shall be regarded as disclosed also for the computer program, the computer-readable medium, the data processing device, the method of generating training data, the method for training a machine learning model for analyzing experimental data, the method for analyzing experimental data by using a machine learning model, the computer-implemented machine learning model, the computer-implemented method for classifying experimental data, the method of identifying an individual based on a specimen of a biofluid of the individual, the method of generating data for analyzing experimental data, and vice versa.

It is understood by a person skilled in the art that the above-described features and the features in the following description and FIGURES are not only disclosed in the explicitly disclosed embodiments and combinations, but that also other technically feasible combinations as well as the isolated features are comprised by the disclosure. In the following, several optional embodiments and specific examples are described with reference to the FIGURES for illustrating the disclosure without limiting the disclosure to the described embodiments.

### BRIEF DESCRIPTION OF THE DRAWINGS

The following disclosure presents further optional embodiments, optional features and possible advantages of the disclosure without limiting the scope of the disclosure.

FIGS. 1A to 9 depict methods according to optional embodiments of the disclosure.

FIG. 10 schematically depicts an overview of problem context and CODI's methodology according to an optional example.

FIGS. 11A to 11D schematically illustrate applying CODI to introduce measurement variability onto illustrative experimental IR spectra.

FIGS. 12A to 12E illustrate CODI enhancing personalized fingerprinting through more accurate long-term molecular profiling.

FIGS. 13A to 13F describe CODI enabling classification flexibility across biological specimen variants.

FIGS. 14A to 14C illustrate CODI recovering lost classification efficacy on independent case-control test sets.

FIG. 15 illustrates the impact of the modeled variability sources on the classification accuracy in longitudinal molecular monitoring

FIGS. 16A and 16B depict a comparison of CODI to domain-agnostic augmentation schemes.

### DESCRIPTION

In the following a detailed description of the FIGURES is presented.

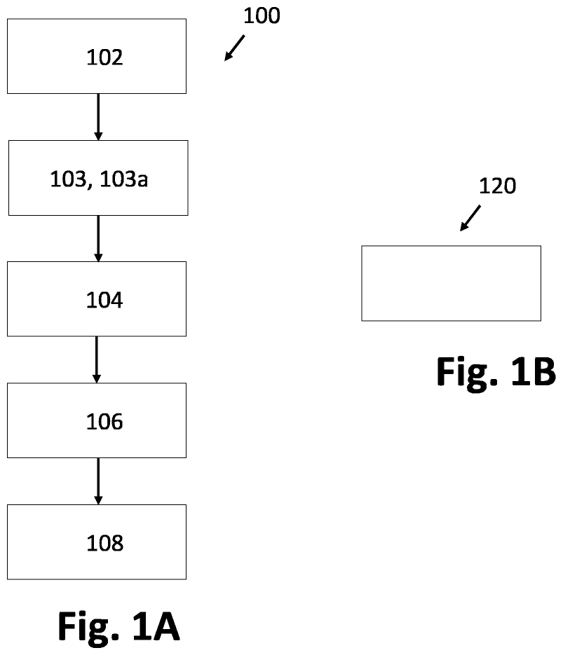

FIG. 1A schematically illustrates a computer-implemented method 100 for analyzing experimental data according to an optional embodiment.

The method 100 comprises receiving 102 a first data set including the experimental data to be analyzed.

The method 100 further includes receiving 104 one or more variation data sets, wherein each variation data set comprises information regarding a variation of experimental observations. The received one or more variation data sets are selected depending on one or more types of noise related to the experimental data to be analyzed. The one or more variation data sets may be selected such that the variation of experimental observations comprised in the one or more data sets correspond to noise typically affecting experimental data of the same type as the experimental data included in the first data set. The received one or more variation data sets may be selected in a contextual manner based on a context related to the experimental data to be analyzed and/or based on a context related to the analysis of the experimental data to be performed.

The method 100 further includes extrapolating 106 the experimental data by applying a variation to the experimental data, wherein the applied variation corresponds to the one or more variations of the experimental observations specified by the information comprised in the one or more variation data sets. Extrapolating the experimental data may comprise or consist of adding a measurement point cloud for at least one measurement point of the experimental data, wherein the measurement point cloud is based on at least one of the one or more variation data sets.

Moreover, the method 100 comprises generating 108 a synthetic data set based on the extrapolated experimental data, analyzing the synthetic data set, and providing an output based on a result of the analysis of the synthetic data set.

The received one or more variation data sets are selected depending on one or more types of noise related to the experimental data to be analyzed.

The received one or more variation data sets may be at least partially selected and/or provided by an external source.

The one or more variation data sets may be selected such that the experimental observations of the one or more variation data sets and the experimental data included in the first data set are of the same type of experimental data.

The method 100 may further comprise, prior to receiving the one or more variation data sets, a step of determining 103 one or more types of noise to be considered when analyzing the measurement data, and selecting 103a the one or more variation data sets to be received based on the determined one or more types of noise.

The experimental observations of the one or more variation data sets may be independent of the experimental data included in the received first data set.

The type of the experimental data may relate to spectroscopic data. The experimental data may include data retrieved by at least one of the following techniques:
vibrational spectroscopy;
optical spectroscopy in the visible and/or ultraviolet spectral range;
X-ray diffraction;
NMR spectroscopy;
mass spectrometry;
clinical chemistry tests;
electrophysiology; and
flow cytometry.

The experimental data may be retrieved from a biofluid of a biological organism. The biofluid may include at least one of the following types:
blood;
plasma;
serum;
saliva;
urine or exprimate urine;
cerebrospinal fluid;
living or chemically fixed animal, human, plant tissue;
living or chemically fixed bacterial, animal, human, plant cells;
viruses or viral particles; and
multimolecular structures.

The variation of the experimental observations comprised in a particular variation data set of the one or more variation data sets may originate in one of the following sources of variation:
a tolerance in quantitative measurement reproducibility of a measurement technique used for retrieving the experimental observations;
a preanalytical variability occurring in a process for preparing one or more samples for retrieving the experimental observations;
a variation between different phenotypes from which the one or more samples for retrieving the experimental observations originate.

FIG. 1B schematically depicts a data processing device 120 according to an optional embodiment, wherein the data processing device is configured to carry out the method according to FIG. 1A.

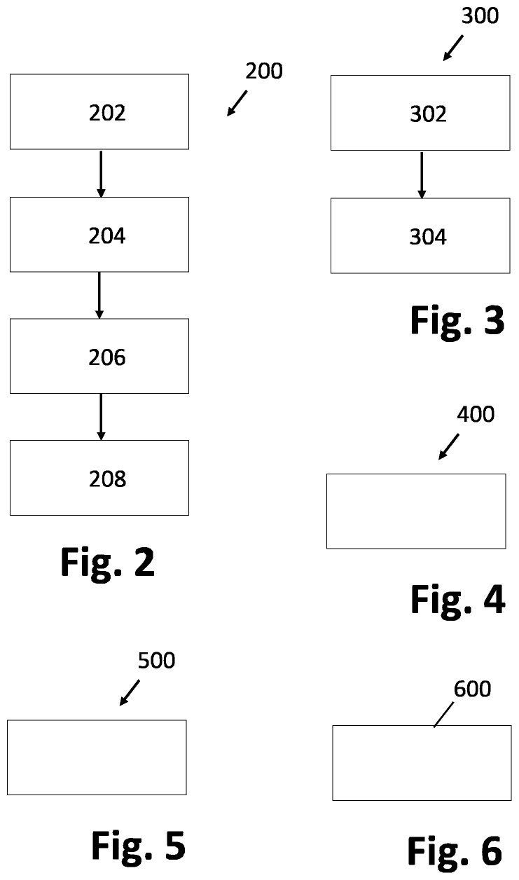

FIG. 2 schematically illustrates a computer-implemented method 200 of generating training data for training a machine learning model to analyze experimental data according to an optional embodiment.

The method 200 comprises receiving 202 one or more variation data sets, wherein each variation data set comprises experimental observations being subject to a variation at least partially affecting the experimental data to be analyzed. The received one or more variation data sets may be selected, such that the variation of the experimental observations comprised in the one or more variation data sets correspond to an actual variation and/or an expected variation affecting the experimental data to be analyzed.

The method 200 further comprises analyzing 204 the variation of the experimental observations comprised in the one or more variation data sets and determining variability properties based on the analyzed variation.

The method 200 further comprises receiving 206 a first data set including at least one reference specimen of the experimental data and a predetermined analysis result for the at least one reference specimen.

The method 200 further comprises 208 generating one or more synthetic data sets including multiple synthetic data points by applying the determined variability properties to the first data set.

The number of synthetic data points may be larger than the number of the at least one reference specimen of the experimental data included in the first data set.

The variation affecting the experimental data to be analyzed may originate in one or more of the following variation sources:
a tolerance in quantitative measurement reproducibility of a measurement technique used for retrieving the experimental data;
a preanalytical variability occurring in a process for preparing one or more samples for retrieving the experimental data; and
a variation between different phenotypes from which the one or more samples for retrieving the experimental data originate;
a variation between types of samples from which the experimental data is extracted.

Each variation data set may comprise experimental observations affected by a variation originating in one or more predetermined variation sources. Alternatively or additionally, each variation data set optionally comprises experimental observations affected solely by a variation originating in one or more predetermined sources of the variation sources.

Each variation data set may comprise experimental observations affected by a variation originating in one or more predetermined variation sources, wherein the experimental observations are essentially unaffected by variations originating in variation sources different from the predetermined variation sources.

The experimental data may include data retrieved by at least one of the following techniques:
vibrational spectroscopy;
optical spectroscopy in the visible and/or ultraviolet spectral range;
X-ray diffraction;
NMR spectroscopy;
mass spectrometry;
clinical chemistry tests;
electrophysiology; and
flow cytometry.

The experimental data may be retrieved from a biofluid of a biological organism. The biofluid may include at least one of the following types:
blood;
plasma;
serum;
saliva;
urine or exprimate urine;
cerebrospinal fluid;
living or chemically fixed animal, human, plant tissue;
living or chemically fixed bacterial, animal, human, plant cells;
viruses or viral particles; and
multimolecular structures.

The reference specimen of the experimental data included in the first data set may be retrieved from the same biofluid type as the experimental data to be analyzed by the machine learning model to be trained with the one or more synthetic data sets. Alternatively, the reference specimen of the experimental data included in the first data set may be retrieved from a different type of biofluid as the experimental data to be analyzed by the machine learning model to be trained with the one or more synthetic data sets.

The one or more variation data sets may be selected such that the experimental observations comprised in the one or more variation data sets are affected by the same and/or a similar type of variation as the experimental data to be analyzed.

The variation of the experimental observations comprised in a particular variation data set of the one or more variation data sets may originate from one of the following sources of variation:
a tolerance in quantitative measurement reproducibility of a measurement technique used for retrieving the experimental observations;
a preanalytical variability occurring in a process for preparing one or more samples for retrieving the experimental observations;
a variation between different phenotypes from which the one or more samples for retrieving the experimental observations originate.

Analyzing the variation of the experimental observations comprised in the one or more variation data sets may include analyzing the variation of the experimental observations of each of the one or more variation data sets independently of each other. Alternatively or additionally, determining the variability properties based on the analyzed variation of the experimental observations may include determining the variability properties based on the experimental observations for each of the one or more variation data sets independently of each other.

Applying the determined variability properties to the first data set may comprise extrapolating each of at least some of the one or more data points of the reference specimen of the experimental data included in the first data to multiple synthetic data points according to the variability properties.

The synthetic data set is provided as training data for training a machine learning model for analyzing experimental data based on out-of-distribution generalization.

FIG. 3 schematically depicts a computer-implemented method 300 for training a machine learning model for analyzing experimental data according to an optional embodiment.

The method 300 comprises receiving 302 a training data set comprising training data, wherein the training data comprises one or more synthetic data sets having the same or a similar variation as one or more variation data sets used for generating the one or more synthetic data sets based on a first data set including at least one reference specimen of the experimental data and a predetermined analysis result for the at least one reference specimen.

The method 300 further comprises training 304 the machine learning model with the received training data set.

FIG. 4 schematically shows a computer-implemented method 400 for analyzing experimental data by using a machine learning model, wherein the machine learning model is configured to classify the analyzed experimental data being subject to a variation exceeding a variation of reference specimen of the experimental data used as a first data set for generating the training data for training the machine learning model.

FIG. 5 schematically depicts a computer implemented method 500 of analyzing experimental data by using a machine learning model, wherein the machine learning model is trained by a method according to FIG. 4.

FIG. 6 schematically shows a data processing device 600 configured to carry out a method according to any one of the FIGS. 2 to 4.

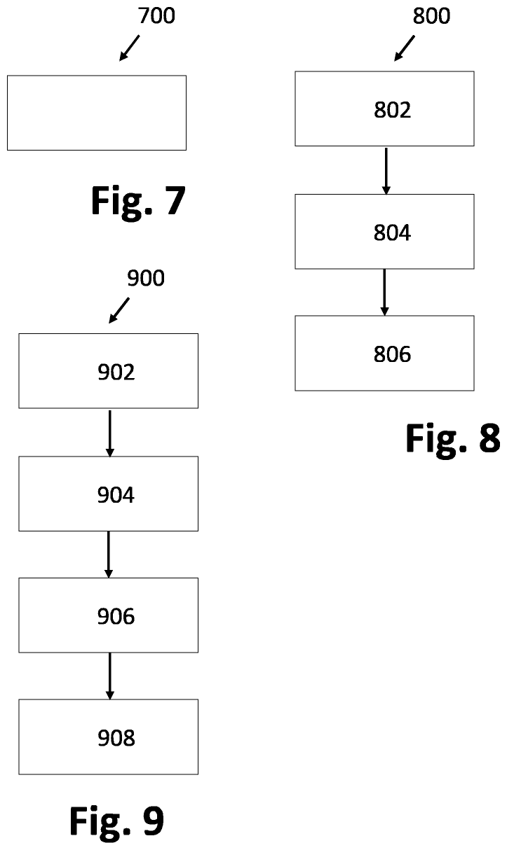

FIG. 7 schematically depicts a computer-implemented method 700 for classifying experimental data being subject to variation using a machine learning model, wherein the variation of the experimental data to be classified exceeds a variation of a reference specimen of the experimental data used for generating the training data for training the machine learning model.

The variation of the experimental data to be classified may correspond to a variation of one or more synthetic data sets used for training the machine learning model, wherein the one or more synthetic data sets are based on a first data set including the at least one reference specimen of the experimental data, a predetermined analysis result for the at least one reference specimen, and on one or more variation data sets comprising experimental observations being subject to a variation at least partially affecting the experimental data to be classified.

FIG. 8 schematically depicts a method 800 of identifying an individual based on a specimen of a biofluid of the individual according to an optional embodiment.

The method 800 comprises receiving 802 experimental data characterizing the specimen of the biofluid of the individual.

The method further comprises determining 804 an analysis result for the received experimental data by using a machine learning model according to an optional embodiment of the disclosure trained on experimental data originating in a reference specimen of a biofluid of the individual, wherein the analysis result corresponds to a match or a mismatch between the received experimental data characterizing the specimen and the experimental data of the reference specimen used for the training of the machine learning model.

The method 800 further comprises providing 806 an information regarding the identification of the individual based on the match or mismatch between the received experimental data characterizing the specimen and the experimental data of the reference specimen used for training the machine learning model.

FIG. 9 depicts a computer-implemented method 900 for generating data for analyzing experimental data according to an optional embodiment.

The method 900 comprises receiving 902 one or more variation data sets, wherein each variation data set comprises experimental observations being subject to a variation at least partially affecting the experimental data to be analyzed.

The method 900 further comprises analyzing 904 the variation of the experimental observations comprised in the one or more variation data sets and determining variability properties based on the analyzed variation.

The method 900 further comprises receiving 906 a first data set including at least one reference specimen of the experimental data and a predetermined analysis result for the at least one reference specimen.

The method 900 further comprises generating 908 one or more synthetic data sets including multiple synthetic data points by applying the determined variability properties to the first data set.

In the following, further optional details and optional examples of the disclosure are described without limiting the scope of the disclosure.

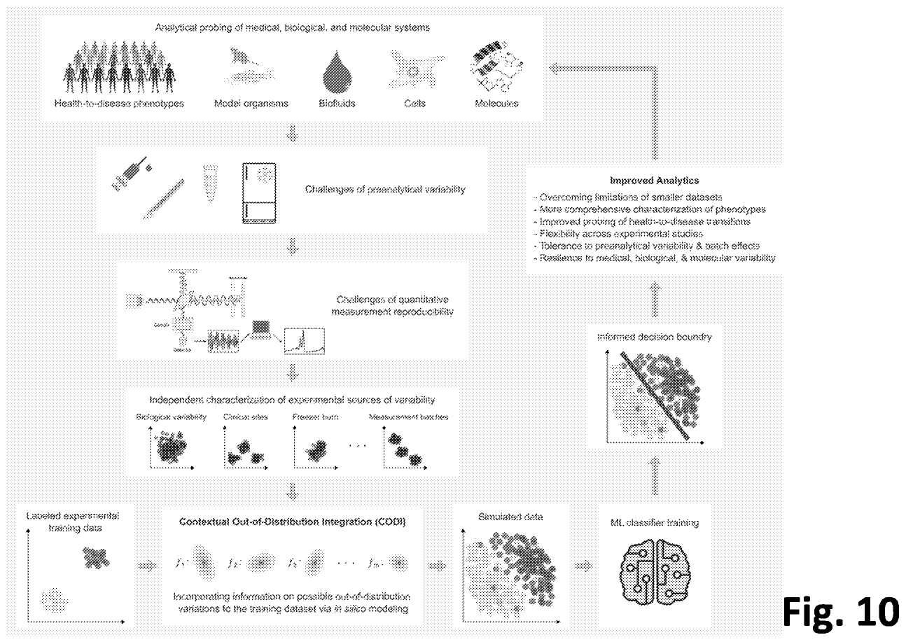

A hybrid experimental and computational modeling framework is provided and empirically tested. We explore OOD generalization in the context of molecular analytics and propose to recognize the variations that arise from the analytical procedure as integral components of real-world observations (FIG. 10). We introduce Contextual Out-of-Distribution Integration (CODI), a modeling framework that paradoxically embraces measurement variability and inherent complexities of biological systems, transforming them into valuable properties that can be utilized. CODI first involves experimental data to evaluate their distributional characteristics. Following the characterization, we deliberately introduce these distributional characteristics into a studied, independent, dataset through the in silico generation of synthetic data. These synthetic data may mimic the system(s) of interest, while expanding the distribution of the original seed data to incorporate information about sources of variance that were crucially OOD and missing.

FIG. 10 schematically depicts an overview of problem context and CODI's methodology according to an optional example. Given a biological, medical, or molecular system of interest, we are presented with a task of classifying distinct groups of samples (e.g., healthy vs. diseased observations). However, variations stemming from several biological and (pre)analytical aspects in the empirical workflow may impact captured measurements in different ways. CODI leverages independently characterized sources of variability and incorporates them into a labeled training set of experimental observations. This process generates a larger set of simulated samples, with a more representative data distribution. Training an ML classifier on simulated samples enables it to learn a decision boundary that separates classes of samples in a more informed manner, increasing the likelihood of generalizing to unseen test samples. This approach may enable sample characterizations that are more robust to variations in the empirical workflow.

To establish the concept and evaluate it in a realistic setting, we apply our method to experimental infrared (IR) spectroscopic data to aid in vitro blood-based diagnostics. The advantage may lie in cross-molecular fingerprinting, where quantitative analytical measurements capture the breadth of changes in the molecular landscape of complex samples as indicators of systemic health and disease. We test our method in the framework of three independent longitudinal clinical studies spanning up to an 8-year follow-up period [25-27], as well as a case-control study to detect four common cancers [28]. Our results demonstrate that integrating CODI into the classification pipeline enables the creation of larger, more representative, datasets that empower ML algorithms to more effectively capture reproducible signals in biological datasets. Ultimately, we showcase how the proposed framework may lead to significantly improved classification output on unseen, independently measured test samples, ensuring robust predictions despite shifts in data distribution.

### Characterizing Empirically Observed Variability

We previously introduced an in silico model that generates 1D spectra of complex biological samples, focusing on IR absorption spectra [29]. Our initial work explored the impact of varying levels of between-person biological variability on classification efficiency in simulated case-control conditions. Building on this foundation, we extend the model beyond the theoretical framework. We generate data simulating longitudinal and/or case-control settings, accounting for diverse sources of possible variability that we experimentally characterize. To assess its practical applications, we explore the capacity of the modeling framework to computationally generate larger training sets that are more robust to biological and analytical variabilities. CODI is a simple statistical procedure. In essence, the method may rely on capturing differences between a set of experimental observations (uᵢ−vⱼ), henceforth called calibration measurements (e.g., a control sample repeatedly measured under varying laboratory conditions). By selecting calibration measurements from a given measurement pool that exhibit a level of variation, we can scale the differences by a random variable assuming a probability distribution, and combine them over the whole set of utilized calibration measurements. This aggregation may then be added onto an independent experimental measurement xₖ to create a new simulated measurement. Such a simulation approach can be repeatedly applied to generate a cohort of measurements in arbitrary size. The generated cohort, as a whole, would reflect the variability properties observed between uᵢ and vⱼ onto xₖ. If the set of calibration measurements would reflect a new source of variability that was unobserved in a given training set of measurements {xₖ|k=1, . . . , n}, a new level of variability would be introduced onto the training set of measurements. This strategy may allow for the creation of realistic synthetic data without requiring the adjustment of any free parameters controlling the data generation—other than the number of generated observations. A detailed description of the modeling approach is provided in the Methods sections and Supplementary Information below.

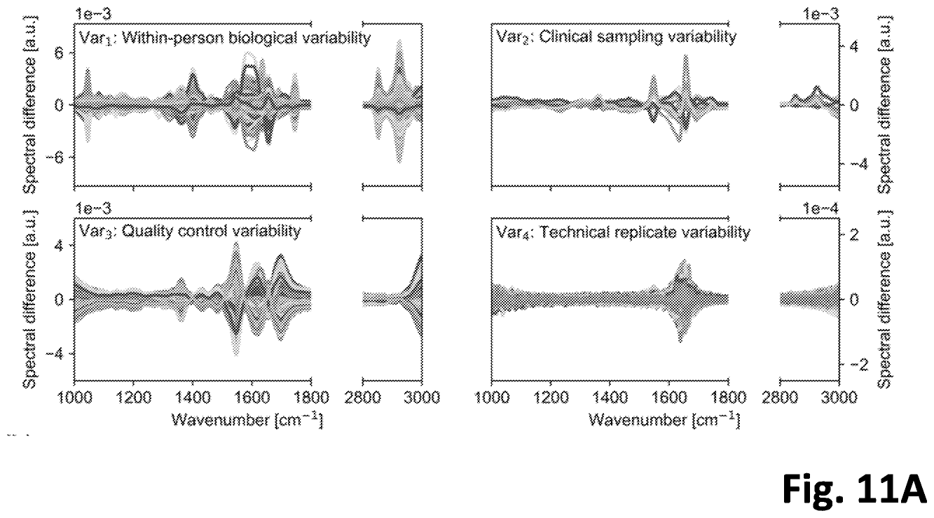

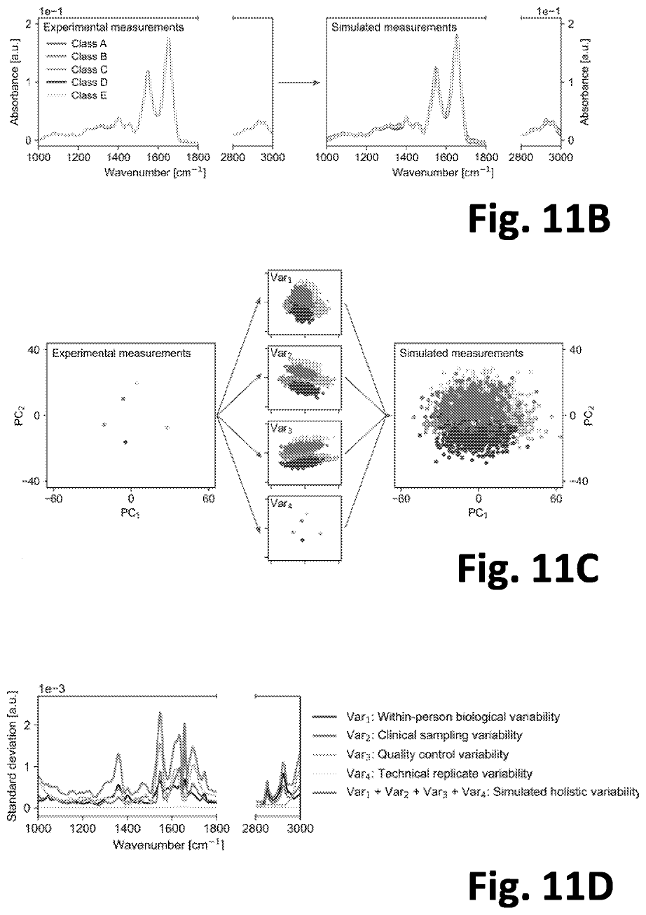

In our example applications of CODI, we introduced several distinct sets of calibration measurements to model different sources of variability that may be observed in IR spectral measurements of blood-based media (FIG. 11A). Within these calibration measurements are characteristics of empirical variability stemming from inherent biological factors, variations in sample collection and handling, as well as instrument-specific measurement noise and drifts (Methods, Supplementary Information).

When addressing biological variability, the calibration measurements uᵢ, vⱼ are selected to be experimental measurements of the same individual over time, capturing a level of within-person biological variability (FIG. 11A, upper left), as defined previously [27]. Alternatively, opting to set the calibration measurements to be of different individuals would yield a level of between-person biological variability, as demonstrated previously [29]. Further exemplary variations that stem from different clinical sample collection sites (e.g., clinics), clinical study protocols, and sample handling procedures may be effectively represented by selecting calibration measurements characteristic of samples derived from different clinical studies (FIG. 11A, upper right). The same concept can be extended to model realistic variations that arise from experimental procedures like sample storage temperature and duration, aliquoting procedures, and measurement device drifts. For example, quality control (QC) samples, may be subjected to diverse handling and storage conditions. Performing measurements of QCs under different operating conditions for the measurement device, including instances of recalibration, routine maintenance, or changes in the surrounding environment would enable the QC measurement dataset to mimic potential variations in both laboratory procedures and instrumental drifts (FIG. 11A, lower left). Further, independent, measurements of technical replicates (e.g., pure water) performed over extended periods can facilitate a clearer distinction between instrumental device noise and laboratory variations (FIG. 11A, lower right).

The overarching goal of characterizing diverse sources of variability is to realistically simulate the data distribution that may be encountered in the empirical workflow. It is crucial to recognize that this is achieved through the utilization of measurements that are independent of the original training set and unrelated to the specific questions posed by it (i.e., class-invariant). When extending the concept to other molecular systems or measurement modalities, similar calibration sets may be adapted to characterize the variations relevant to the studied conditions.

Introducing experimental variability in silico as an illustrative example, we applied the CODI modeling framework to five experimental spectra of blood plasma spectra to generate a larger set of simulated measurements that reflect an increased level of variance (FIG. 11B). The five original spectra may be considered to be a training set, with each measurement representing a labeled class (FIG. 11B, left). Using the five measurements as a seed input, the CODI framework allowed us to generate a larger and more representative training set of measurements (FIG. 11B, right). Principal component analysis (PCA) applied to the original and simulated measurements reveals that each source of measurement variability affected the spatial distribution of the seed data differently across the first two components (FIG. 11C). In other words, each set of calibration measurements—modeling different variability properties—affected linearly independent data features. Once all four sources of measurement variability were incorporated into the original seed data (FIG. 11C, right), the simulated measurements occupied a larger cloud of data points, while still maintaining their distinct cluster centroids. Similarly, an examination of the standard deviation between each source of modeled empirical variability and the simulated measurements reveals that the standard deviation of simulated measurements was higher than that of each independent source of empirical variability (FIG. 11D). While it may seem counter-intuitive that a simulated dataset with increased variance could offer added value compared to the existing experimental observations, this variance contains valuable, usable information. The principle relies on the assumption that the simulated measurements represent unaccounted fluctuations in a smaller set of measurements, out-of-distribution, that are likely to occur when a larger set of experimental observations is available.

FIGS. 11A to 11D schematically illustrate applying CODI to introduce measurement variability onto illustrative experimental IR spectra. FIG. 11A: Four distinct sources of possible variability were characterized from calibration sets of experimental observations. FIG. 11B: By applying CODI, the four sources of variability were introduced to five illustrative experimental blood-based spectra (left panel) to generate a larger set of simulated spectra (right panel). FIG. 11C: Principal component analysis (PCA) on the five illustrative experimental spectra (left panel), on a larger simulated set of spectra that introduced only one out of the four characterized sources of variability (middle panel), and on a simulated set of spectra that introduced all four sources of variability to the five illustrative experimental spectra (right panel). FIG. 11D: Comparison of the standard deviation across the fingerprint spectral range for each characterized source of variability and the standard deviation of simulated measurements (incorporating all four sources of variability), resulting in an overall increased standard deviation.

So far, we have shown how CODI can be used to introduce additional sources of variability into an existing measurement dataset to enrich its information content. The value of the method for other applications is examined in the following sections.

### Application to Longitudinal Study Settings

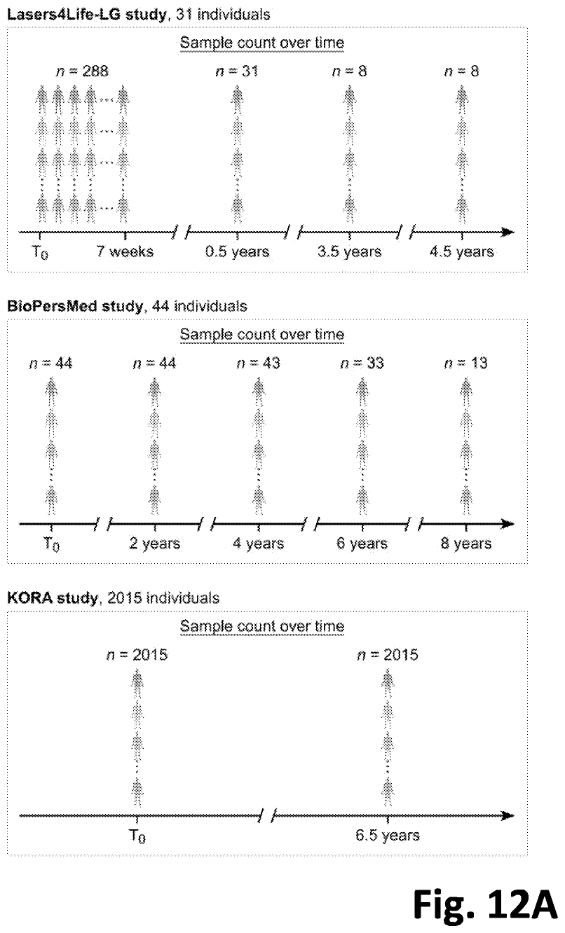

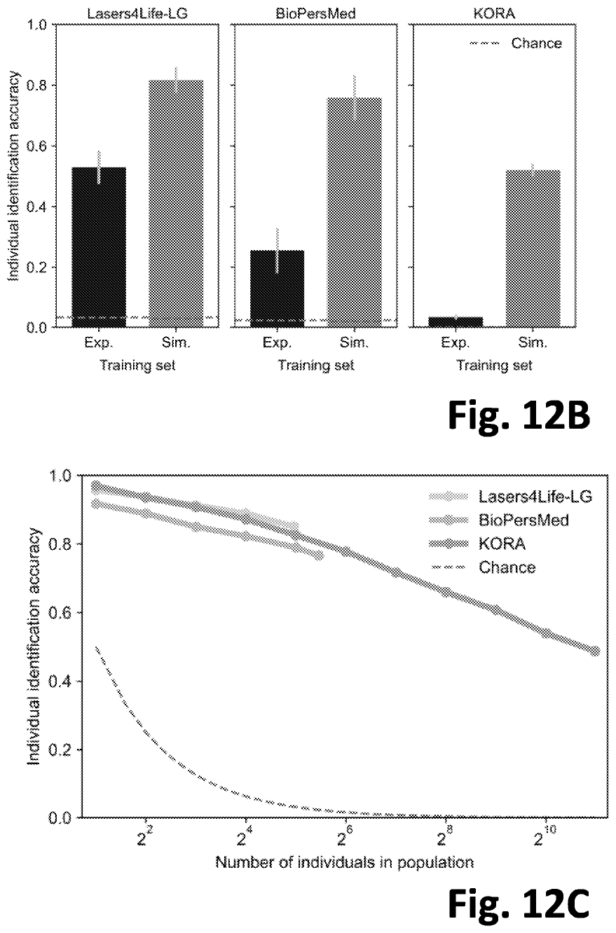

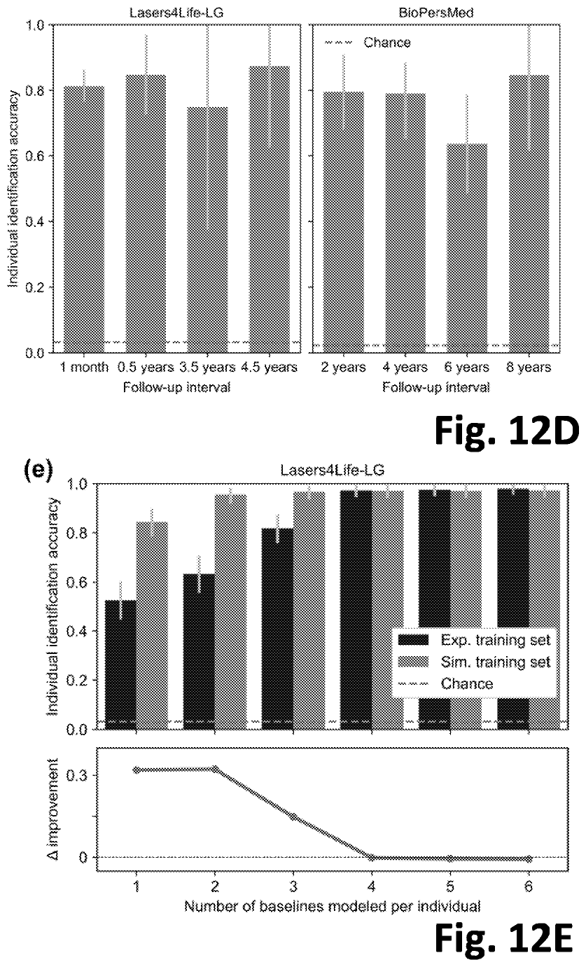

In longitudinal clinical studies that involve the collection of biological specimens tainted by attrition and loss-to-follow-up over time, great efforts may be required to gather sufficiently large datasets. Typically, individuals participate in an initial baseline sample collection, followed by extended waiting periods for subsequent collections from the same individuals. In situations where only few samples are initially available per individual, the challenge arises in extrapolating meaningful insights to later collected and measured follow-ups—owing to the dynamic nature of the empirical procedure as previously described. To examine whether our proposed approach offers added value when severely limited samples are available for analysis, we employ the CODI framework in the context of longitudinal analyses (FIG. 12A-E).

We first utilized three independent clinical studies that followed individuals over extended periods (FIG. 12A). The Lasers4Life-LG study cohort [27] comprised of 31 individuals that repeatedly donated blood samples at irregular follow-up intervals. The study commenced with a 7-week baseline monitoring period, during which 288 samples were collected through repeated donations. The initial baseline donation period was followed by three additional donations, spanning up to 4.5 years, during which one sampling point was considered per individual. In the BioPersMed study [25], a subcohort of 44 individuals repeatedly participated over an 8-year follow-up period, with a 2-year interval between each donation. In the KORA study [26], a subcohort of 2015 individuals participated in two donations, separated by a 6.5-year follow-up interval. Blood plasma was processed from all samples and measured via absorption IR spectroscopy (Methods).

FIGS. 12A to 12E illustrate CODI enhancing personalized fingerprinting through more accurate long-term molecular profiling. FIG. 12A: Setup of three independent longitudinal clinical studies in which same individuals repeatedly participated in venous blood sampling over time. Experimental IR spectroscopic measurements were performed on blood plasma and utilized in this analysis. FIG. 12B: Individual identification efficacy utilizing only a single baseline IR measurement per individual across the three study cohorts. Black bars depict the classification accuracy using experimental (“Exp.”) baseline measurements for training. Blue bars depict accuracy using simulated (“Sim.”) measurements generated by CODI using a baseline IR measurement per individual as a seed input. Classifier testing was performed on the same experimental follow-up measurements of each individual for both training approaches. FIG. 12C: Dependence of identification accuracy on number of individuals in the populations (i.e., number of classes). Individuals were randomly selected, at varying population sizes, and CODI was applied using a single baseline IR measurement per individual as a seed input. Classifier testing was performed on experimental follow-up measurements of each individual included in the training set. FIG. 12D: Dependence between the identification accuracy and the follow-up time axis on applications involving the Lasers4Life-LG and BioPersMed cohorts. FIG. 12E: Modeling an increasing number of experimental baseline measurements per individual as a training seed. The bars on the right-hand side depict the identification accuracy on simulated training sets, while black bars depict the identification accuracy on experimental training sets. Model testing was conducted on the same experimental follow-ups beyond the first six baseline measurements per individual. The lower panel illustrates the difference between accuracy observed from the experimental and simulated training sets.

### Empowering Long-Term Molecular Profiling

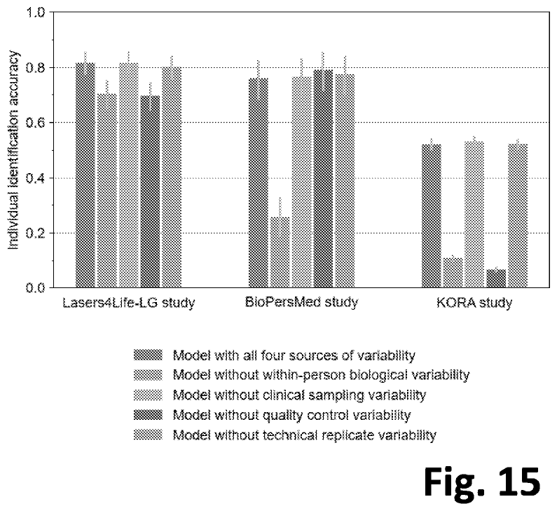

The concept of identifying individuals based on different biofluids has been demonstrated with IR spectroscopy, NMR spectroscopy, and mass spectrometry as fingerprinting modalities [27, 30-32]. We previously demonstrated that plasma- and serum-based IR fingerprints can identify individuals over a span of 6 months [27]. Such an application inherently relies on the stability of measurements over a study period, often requiring the comparison of measurements acquired at different times despite inevitable experimental drifts [33]. In previous works [27, 30-32], several measurement data points from the same individuals were required to adequately train a multi-class classifier to identify individuals (typically 8-42 samples). We set out to test the possible value of our CODI framework for enhancing longitudinal studies, using the individual identification task as a readout metric. To test the limits of the framework, here we considered a scenario with severely limited training data relying only upon a single experimental observation per class for training, i.e., one measurement per individual (FIG. 12B). We utilized the first baseline measurement of each individual to train a multi-class classifier to identify individuals from their follow-up measurements. We then compared this prediction efficiency to a classifier trained on a simulated set of measurements created through the CODI framework that used the experimental baselines as initial seed data. Within the CODI framework, we modeled the four previously described sources of variability (FIG. 11A), which were characterized from data sources independent of the experimental seed data (Supplementary Information). This allowed us to generate 1000 simulated measurements per individual that were used for training the classifier. This investigation revealed that the classifier trained on simulated measurements had a remarkably improved prediction efficiency compared to the classifier trained on experimental measurements (FIG. 12B). Across the three cohorts, the individual identification accuracy improved from 0.53 to 0.82, from 0.26 to 0.76, and from 0.03 to 0.52. This demonstrated that the (informed) incorporation of data variance was indeed capable of enabling the classifier to better generalize to unseen test samples. To examine which sources of data variability aided the most in boosting the classification efficiency, we re-performed the above analysis, but systematically eliminated one of the four sources of variability incorporated in the CODI modeling framework (FIG. 15). We found that the within-person biological variability over time and measurement variability of the same quality control samples over the course of measurements were the most critical contributors for the success of the classification. Including the variability stemming from technical replicates and clinical sampling had minimal impact on the classification, compared to the two aforementioned sources. It is emphasized that the population size varied between the cohorts. The decreased accuracy observed in the KORA cohort (involving 2015 individuals) is thus not directly comparable to that of the Lasers4Life-LG cohort (involving only 31 individuals). This is because the more individuals exist in a dataset, the more likely it is that their fingerprints will overlap with one another—making the task of identifying individuals more challenging. Despite this, it was very surprising and encouraging to observe that nearly half of 2015 individuals can be identified from IR molecular fingerprints when combined with the proposed modeling approach—and requiring only the venous blood sampling of only a single baseline sample per individual. Observing that the identification accuracy decreased with an increasing population size prompted us to further investigate this dependency (FIG. 12C). We first trained a classifier on simulated measurements utilizing the first experimental baseline measurements of only two individuals and, as previously, tested on their follow-ups. This procedure was repeated several times, using two other randomly selected individuals. We then performed the same procedure, but on 4, 8, 16, and so on individuals. This analysis revealed that the identification accuracy, depending on the population size, follows a nearly perfect logarithmic trend. This intriguing finding draws from information theory and may be explored further to quantify the informational content of diverse molecular fingerprints. Remarkably, these results were reproducible on three independent cohorts, revealing that a similar identification accuracy can be achieved when the datasets involve similar population sizes. In the above application, follow-up measurements of all individuals were pooled together and the accuracy of identification was averaged, independent of the follow-up time axis. This prompted the question of whether the individual identification accuracy was dependent on the time interval between follow-up measurements and the baseline. In other words, is it more difficult to identify an individual 8 years after their baseline sample was assessed than from a 2-year follow-up? To investigate this, we grouped the follow-up measurements by their time differences to the baseline and examined whether any temporal trend was observed in the identification accuracy (FIG. 3d). This analysis was only possible on the Lasers4Life-LG and BioPersMed cohorts, since the available KORA cohort only involved one follow-up. Here, we revealed that the identification accuracy did not depend on how far off the follow-up was from the baseline. The accuracy remained relatively stable even over an 8-year follow-up period. Although it is crucial to recognize that the number of test samples in the later follow-up years was limited (FIG. 12A), this is the very first experimental result over such long-lived fingerprint stability. Altogether, the above investigations were made possible by using the CODI framework which enabled applications that were previously unfeasible with limited experimental observations.

### Comparison to Domain-Agnostic Augmentation Schemes

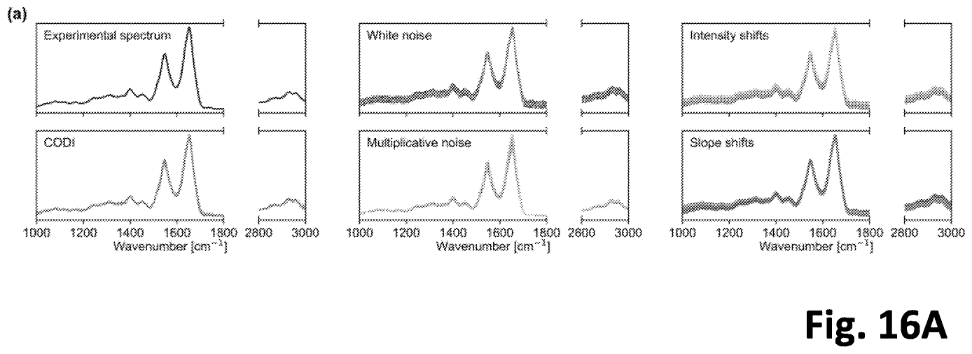

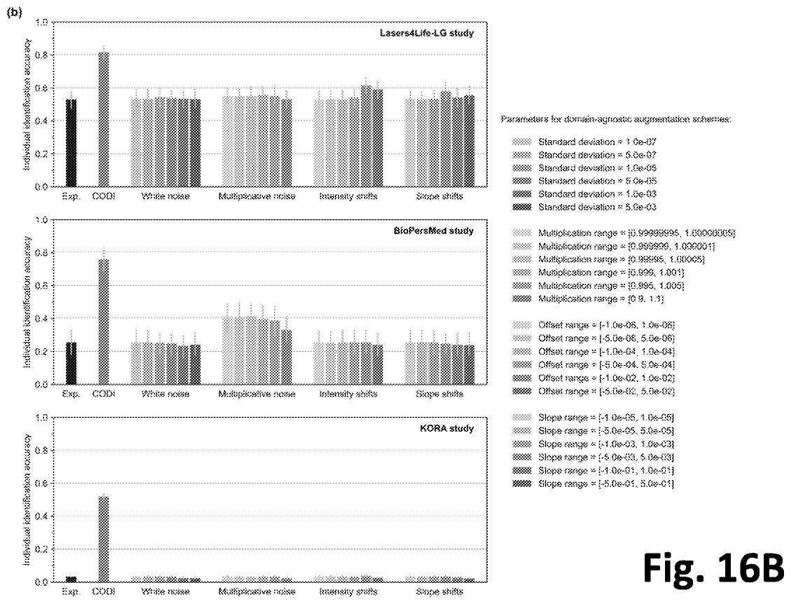

The CODI modeling framework inherently relies on a priori information on potential sources of measurement variability. In contrast, domain-agnostic augmentation methods employ generic transformations on input seed data to simulate new observations (e.g., introducing additive or multiplicative noise). By eliminating the need for a priori information, such a strategy is simpler to implement than CODI. To examine whether CODI yielded an advantage over other augmentation methods, we re-performed the above analysis by applying several methods of augmenting the spectral measurements (Supplementary FIGS. 16A and 16B). We found that the CODI method of generating training sets significantly outperformed all other methods of data augmentation. This underscored the value of incorporating contextual a priori information into the data augmentation process to enable the classification to generalize beyond the original training set.

### Personalized Multi-Baseline Modeling

In the above quest of examining the value of the CODI framework, we relied on a single baseline measurement per individual. We then questioned to what extent the classification can be made more robust when more training instances per class are available. Specifically, considering that a single baseline measurement may be an outlier, we examined the dependence between the number of training instances per individual and the identification accuracy (FIG. 12E). For this analysis to be properly investigated, individuals would have to be repeatedly sampled in each baseline monitoring period. Among the three clinical studies, only the Lasers4Life-LG study facilitated such a setup [27]. Here, we utilized data from up to the first 6 baseline measurements per individual from the Lasers4Life-LG cohort to be used as an experimental seed for training. Then, we simulated a training set that consisted of 1000 measurements per baseline and investigated how the identification accuracy depended on how many experimental baselines were modeled per individual.

Classifier testing was performed on the remaining follow-ups that were beyond the first 6 baselines of each individual. This investigation revealed that the identification accuracy following the simulation-based approach could indeed be improved when more than one baseline measurement was modeled per individual (FIG. 12E, bars on the right-hand side). When using only one baseline measurement, an accuracy near 0.85 was achieved. Surprisingly, including only one additional baseline already led to an improvement of a nearly perfect prediction efficiency, achieving an accuracy of 0.96. As a comparable benchmark, we again examined the dependence between the identification accuracy and the number of baselines modeled per individual, but now training the classifier directly on the experimental measurements (FIG. 12Ee, bars on the left-hand side). The classifier was first trained on one experimental baseline per individual, then again on two, and up to 6 baselines of each individual. Testing was performed as previously—on the remaining follow-ups beyond the first 6 training baselines per individual. Here, it was again revealed that the simulation-based approach had a significant advantage over the experimental modeling approach—but only when few observations per class were available (3 baselines per individual). Once ≥4 experimental baselines per individual were available for training, the experimental modeling approach had also achieved a near perfect prediction efficiency and thus no advantage was seen by applying CODI. This underscored the impact of our proposed modeling paradigm in contexts with only limited experimental datasets. Once sufficiently large experimental data sets are available, the simulation-based training approach may not provide an advantage over training directly on experimental data. Altogether, these findings show that the CODI framework can enable the establishment of a more reliable “baseline” per individual—one that is more resilient to analytical and biological variations and can more robustly enable ML generalization. We further demonstrate that IR molecular fingerprints are highly stable and individual-specific. Previously, this was only demonstrated on the timeframe of 6 months [27]. In the current study, we extend these findings to a medically relevant time frame of 8 years. These results form the foundation for future applications of blood-based IR fingerprinting as a modality of personalized monitoring of human health over time, potentially requiring as few as one or two baseline samplings.

### Cross-Specimen Generalization

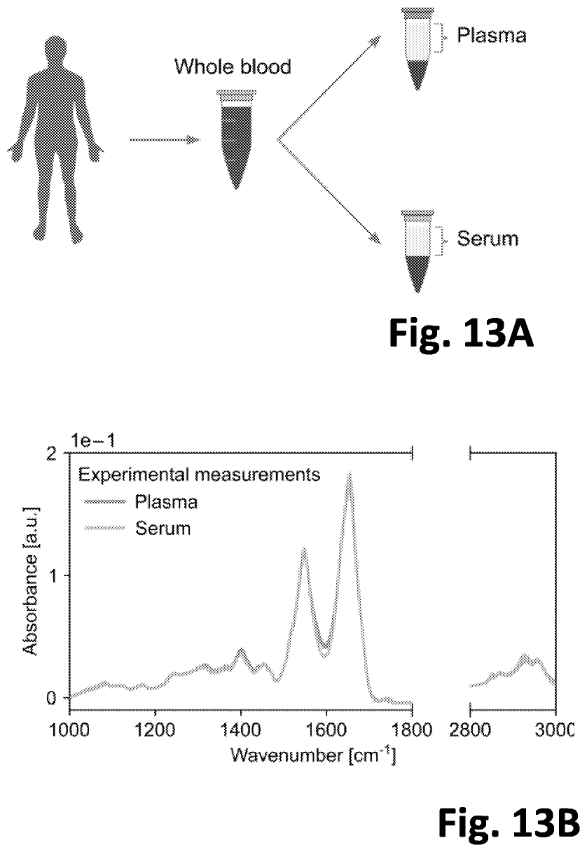

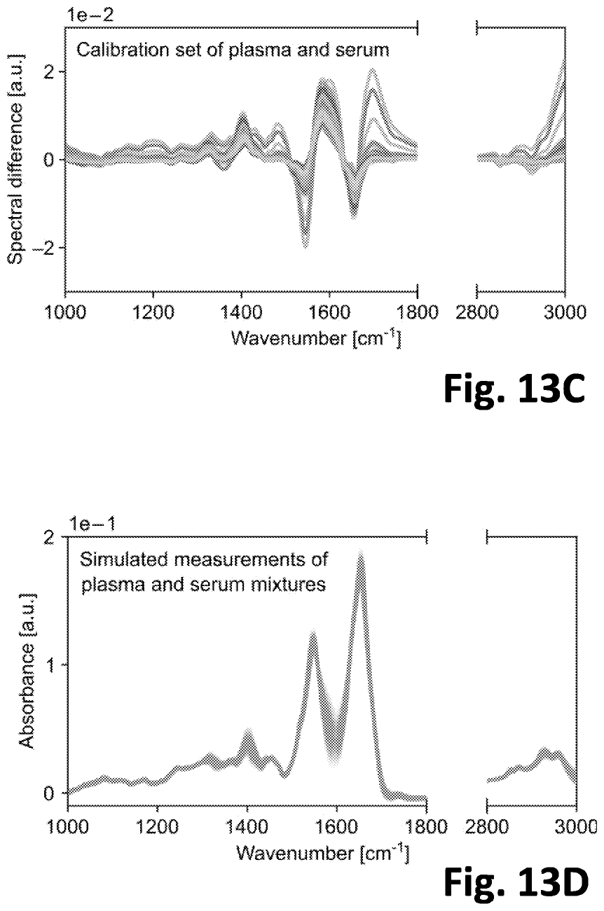

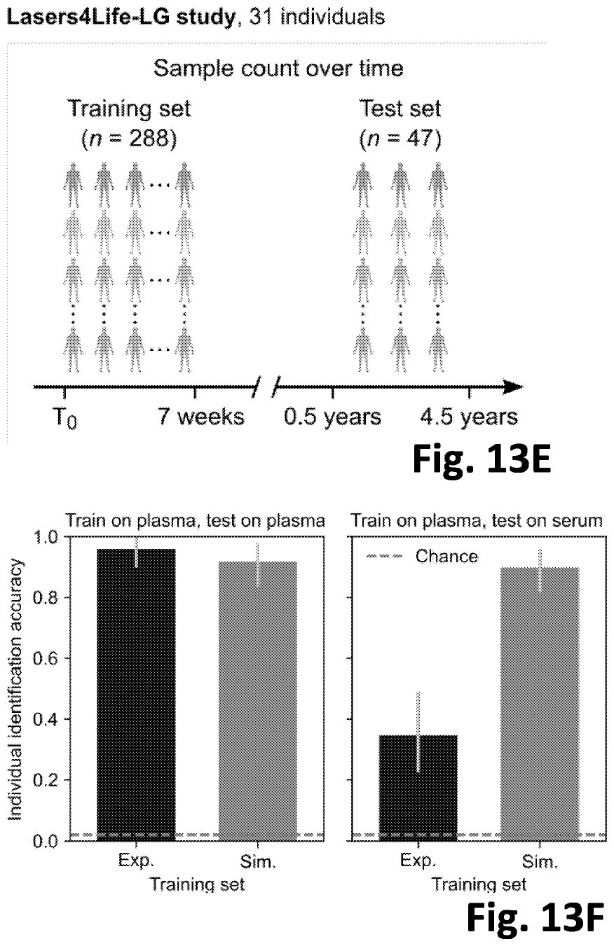

Molecular profiling applications involve the use of diverse sample specimens—e.g., serum or plasma as cell-free products of systemic blood (FIG. 13A). Selecting an appropriate specimen is typically made in a study design phase, considering factors like ease of collection and biological relevance [34, 35]. However, limitations may arise from a preemptive selection as insights gained from one specimen may not generalize when transferred to another. For instance, assume a dataset of plasma spectra is available. Later, the need arises to classify and compare unlabeled spectra that originate from serum samples. This prompted an intriguing question: how well would a classifier trained on plasma spectra perform when tested on serum spectra? The straightforward answer is that the classification is likely to fail, due to underlying molecular differences between the specimens [36]. Effective classification necessitates the inclusion of training instances from different specimens, each with sufficient representation to capture class-specific distributions—a highly resource-intensive process. As proof-of-principle, here we demonstrate the potential versatility of the CODI framework to enable such an application, while minimizing the need for extensive biological dataset collection.

The IR spectra of plasma and serum share many characteristics, due to their relatively similar molecular profiles (FIG. 13B). The main variations stemmed from the plasma preparation process, which involved the use of ethylenediaminetetraacetic acid (EDTA), as demonstrated in previous work [27]. To achieve effective classification flexibility between specimens, their differences must be well-characterized. This can be achieved by calculating differences between experimental plasma and serum measurements of the same collected blood sample (FIG. 13C). Following the CODI framework, we incorporated such characterized differences into an independent experimental dataset of plasma spectra to generate simulated spectra that resemble a mixture between the specimens (FIG. 13D). Next, we revisited the task of identifying individuals as a read-out metric to estimate the capacity of cross-specimen generalization. We employed the Lasers4Life-LG cohort (FIG. 13E), where both serum and plasma were available from the same individuals at all blood sampling donations. The dataset was split into a training set, consisting of 4-12 donations per individual, and a test set, consisting of the remaining follow-up donations. As a benchmark, we first examined the efficacy of training two classifiers—one trained on experimental plasma spectra and one trained on simulated plasma spectra, testing both on plasma spectra (FIG. 13F, left panel). This investigation confirmed the above-mentioned discovery—in that, with sufficiently large experimental training sets, both experimental-based and simulation-based classifiers perform similarly. We then applied the same classification procedure as above, but here testing on serum measurements (FIG. 13F, right panel). For this analysis, we employed the CODI framework to generate a training set of plasma/serum mixtures. Remarkably, we found that the simulation-based approach had a significant advantage over the experimental approach. A substantial drop in prediction efficiency was observed for the classifier trained on experimental plasma, achieving an accuracy of 0.34. The classifier trained on simulated mixture data nearly fully recovered the initial classification efficiency—achieving an accuracy of 0.89. This unexpected finding demonstrated that the CODI framework enabled the creation of a dataset that can even be robust to variations in biological specimen characteristics.

Altogether, this proof-of-principle analysis further demonstrated the CODI framework's potential to overcome conventional limitations in biological and biomedical research, enabling new applications. For one, there is no need to re-collect a large number of specimens when deviations occur in the sample collection procedure. One can leverage a limited set of measurements that characterize differences between specimens, collected in a class- and task-independent fashion. The CODI framework may then be employed to extend ML applications to different specimen variations. Nevertheless, here we only demonstrated such potential on serum and plasma. If the specimens widely vary in their molecular composition and reflection of physiology (e.g., blood-based vs. urine- or saliva-based media), this approach may not perform as effectively as demonstrated here. A promising avenue for future exploration may involve adapting a classifier trained on EDTA plasma for use with citrate samples [37].

FIGS. 13A to 13F describe CODI enabling classification flexibility across biological specimen variants. FIG. 13A: Plasma and serum were collected at the same sampling point as cell-free products of whole venous blood. FIG. 13B: Experimental spectra were measured from several plasma and serum samples of the same individuals. FIG. 13C: Differences between spectra of plasma and serum, processed from the same whole blood sample, were calculated to reveal the characteristic variations between the specimens. FIG. 13D: CODI framework enabled the creation of simulated spectra of plasma/serum mixtures by utilizing the characteristic variations between the specimens as a calibration set. FIG. 13E: Setup of Lasers4Life-LG cohort in which the same individuals repeatedly participated in venous blood sampling over time. Donations were split into a training set and a test set, with blood plasma and serum processed from all donations. FIG. 13F: Individual identification accuracy utilizing the Lasers4Life-LG cohort as a basis for training and testing. Left panel depicts the accuracy of classifiers trained on experimental plasma fingerprints and simulated plasma fingerprints—testing both classifiers on experimental plasma fingerprints. Right panel depicts the accuracy of a classifier trained on experimental plasma fingerprints and simulated fingerprints of plasma/serum mixtures—testing both classifiers on experimental serum fingerprints.

### Generalization to Independently Acquired Datasets

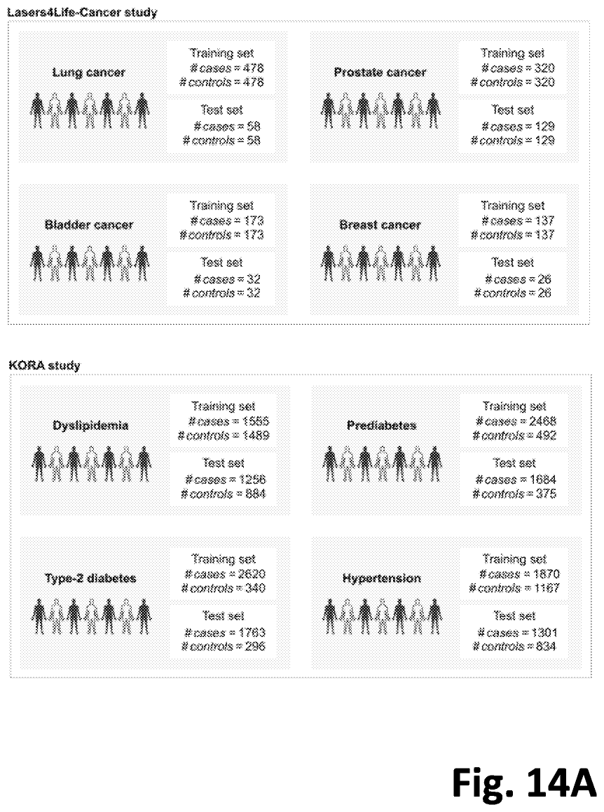

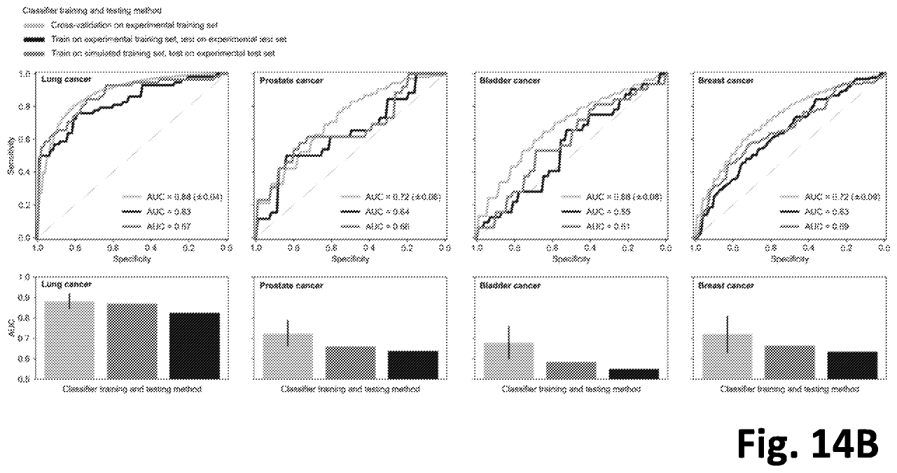

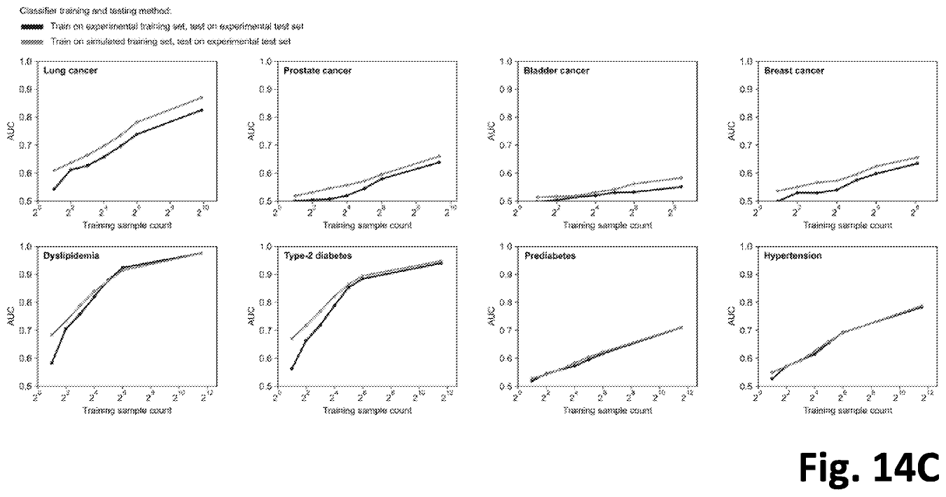

An important aspect in determining how well a medical diagnostic assay is likely to perform is to test it on unseen samples. In biodiagnostic applications, a cross-validation procedure is commonly applied to get an estimate of true (external) classification performance. However, if a bias exists in the collected dataset, e.g., confounding information caused by measurement “batch effects”, the estimated performance may not be reproduced when the classifier is truly externally validated [38]. To test this in a relevant medical application, we considered our previous work [28]—in which four cancer entities were classified against non-symptomatic cancer-free controls. In contrast to our prior work which employed cross-validations [28], here the samples were initially split into a training and a test set that were measured independently (FIG. 14A, left panel). As a benchmark, we first investigated the performance of classifying each cancer entity, relying on a cross-validation procedure (FIG. 14B, left-hand side bars). The cross-validation was performed exclusively on the training set of experimental samples and the receiver operating characteristics (ROC) curve of the validation splits was examined for each cancer entity. Next, we trained a classifier on the training set of experimental samples, testing it on the test set of experimental samples (FIG. 14B, right-hand side bars). This investigation revealed that the classification efficiency across the four cancer entities decreased when tested on the later-measured samples. This validated the prior notion that the cross-validation estimates could not be entirely reproduced with this train-test split classification setup.

Next, we questioned whether the CODI framework can aid in such a scenario. In principle, by introducing class-invariant empirical variability into a training set of measurements, we can practically make the learning task more difficult for the classifier. Potentially, this would enable the classifier to appropriately weigh features that are more robust to measurement artifacts, making it rely on information that is likely to be reproduced in unseen data. To test this, we employed the CODI framework to introduce an additional level of variance to the training set of measurements (Supplementary Information). Across the four cancer entities, we revealed that an improvement in prediction efficiency was indeed observed when testing the classifiers on the held-out test sets (FIG. 14B, center bars). The most impressive improvement was for the lung cancer application, where the area under the ROC curve (AUC) was nearly fully recovered and was comparable to the prior cross-validation estimate. For the remaining cancer entities, the CODI framework still provided an advantage, though not to the same extent as with lung cancer. This may be partly attributed to the occurrence of measurement artifacts in the training set that happened to correlate with the outcome of interest, leading to an overly optimistic AUC estimate during cross-validation. It may also be, in part, due to the generally smaller sample sizes used for testing the classifier and the randomly selected test samples included cases and controls that were more difficult to distinguish than those in the training set (e.g., due to inherent physiological variations that interfere with the cancer signals). Nevertheless, compared to directly training on experimental observations, the CODI framework consistently led to improved classification output on independently measured test sample data.

Influence of experimental training cohort size to investigate under what conditions the CODI framework enables more robust classifier training, we repeated the above case-control investigations, but varied the number of experimental observations utilized for training (FIG. 14C). In addition to the previous cancer applications, here we also involved case-control applications from the KORA cohort [39]—focusing on detecting common health physiologies (FIG. 14A, right). We randomly selected samples at varying cohort sizes to train several classifiers on each subset of selected samples (Supplementary Information). First, we trained directly on the experimental observations, always testing on the held-out test sets (FIG. 14C, black curves). Unsurprisingly, the smaller the training set was, the worse the classifier performed on the experimental test sets. We then employed the CODI framework to generate simulated datasets that utilized the experimental observations at each sample count as seed input (FIG. 14C, gray curves). Here, it was revealed that CODI almost consistently outperformed the experimental modeling approach across the varying sample counts available as a basis for training. Notably, for the detection of dyslipidemia and type-2 diabetes, two conditions with strong molecular deviations reflected in IR fingerprints, CODI provided the largest advance when smaller training sample counts were available. For the detection of prediabetes and hypertension, no clear advantage was observed by incorporating CODI into the classification workflow. This could either stem from the experimental training data already closely resembling the test data distribution, or because the variability introduced by CODI failed to effectively capture the distribution shifts present in the test data. While no advantage was observed for these two conditions, including CODI did not have an adverse impact on the classification. This observation suggested that integrating CODI into the classification pipeline may be an effective standard practice—as it either enhances prediction performance or, at minimum, does not impair it.

Altogether, our findings show exciting promise for the proposed CODI framework. The value of the method has been demonstrated in the context of several biomedically relevant applications, where the method achieved improved ML classification output for several practical applications.

FIGS. 14A to 14C illustrate CODI recovering lost classification efficacy on independent case-control test sets. FIG. 14A: Setup of eight binary classifications, spanning diverse health conditions, in two independent clinical studies. IR spectroscopy of blood plasma was performed on all samples. Training and test sample sets were measured in different measurement campaigns under different measurement device conditions. FIG. 14B: Cancer detection was investigated under three different setups of estimating classification efficiency. For the simulation-based training, the CODI framework was employed to introduce measurement variability into the training set measurements. ROC curves are depicted for the validation splits of the data (upper panel), along with the estimated AUCs (lower panel) for each cancer detection application. FIG. 14C: Classification efficiency when training a classifier on experimental observations (black curves) and applying the CODI framework to train a classifier on simulated data (gray curves) utilizing varying training sample counts as a basis for training. Classifier testing was performed exclusively on held-out experimental observations.

### DISCUSSION

Multimolecular profiling and computational modeling offer promising avenues to advance our understanding of biological systems. In this study, we introduced CODI, a modeling framework designed to enrich collected datasets to facilitate robust analytics for enhanced levels of system probing. Across several experimental settings, we rigorously tried and tested the framework within the context of molecular profiling to demonstrate its validity. We examined how different empirical and biological factors led to variations in data measured through IR spectroscopy, and revealed the framework's advantage in overcoming the limitations of unrepresentative observational datasets. Effectively, the datasets generated through CODI enable an ML algorithm to better capture latent information that is present in a studied dataset, guiding it to distinguish what features are most relevant and reproducible. Such a strategy is particularly valuable when collecting large, representative datasets presents a limitation. This is exemplified in the context of studying pathophysiological phenomena through molecular profiling (e.g., omics). In such instances, biological experimentation or medical studies demand substantial involvement, including the probing of a significant number of subjects, considerable time for phenotypic evolution, and the added constraints of intricate sample collection and handling [1-4, 40, 41]. Another layer of complexity comes from the fact that biological variations at an organismal level are inherent and inevitable—due to the dynamics of biological systems and human physiologies (e.g., recycling, turnover, rhythmic oscillations, aging) [4, 42-46]. These challenges are further compounded by the often-involved quantitative measurement procedures. Factors like the wear and tear of measurement device components, routine maintenance, and sensitivity to environmental conditions all may lead to known “batch effects” that are often specific to a quantitative analytical approach [5, 6, 13, 47, 48]. Ultimately, these challenges impede the generalization of insights to unseen, later collected and measured samples. The concept of CODI to facilitate generalization draws on data augmentation techniques, which rely on modifying existing experimental observations to create synthetic data. Such techniques find widespread applications in image classification, where data augmentation includes geometric transformations (e.g., rotation, skewing, cropping), color adjustments, and introducing random noise into a training set of images to capture potential variations in practical applications [23]. Data augmentation has also been applied to measurements of biological signals from electroencephalography (EEG), electromyography (EMG), Raman, and near-IR spectra [49-56]. While existing methods often involve random noise introductions, signal warping, and decomposition of available datasets, our approach extends the concept of data augmentation. We take advantage of additional, independent measurements that were not initially a part of the original dataset of interest to model real empirical variations for the involved processes of molecular analytics.

In our investigations, we addressed the topic of barely supervised learning [57]—where the set of labeled training samples is limited to very few observations per class. Given the experimental constraints of populational sampling over year-long timeframes, we examined whether the number of sequential samplings of the same individual over time could be minimized with CODI. With the example of IR fingerprinting, we surprisingly identified that only a single baseline measurement is required to sufficiently follow-up and identify an individual in a population at a later time point. Although identification of the same individual in a heterogeneous population is only a distant approximation to identifying physiologically relevant deviations, it presents a foundational addition to the concept of longitudinal probing. Our generic framework can be quickly adopted to possibly spare unnecessary samplings and thus inform future prospective studies.

Further applications of CODI to IR spectroscopic fingerprinting showcased its versatility and potential impact in aiding data analytics. We observed a remarkable level of comparability across experimental data collected over almost a decade, underlining the method's ability to boost classification efficacy on independently measured test sets. The adaptability of the framework extended to a proof-of-principle application that involved training a classifier on one sample medium (plasma) and applying it to another (serum). This application demonstrated the potential of the CODI framework to streamline cross-specimen dataset analyses in various biological and biomedical applications. Such a strategy may be particularly valuable when gathering data sets from retrospective studies or online repositories to help ensure specimen comparability to another envisioned application—i.e., domain adaptation applications [58, 59]. Another promising use of CODI may be to harmonize data obtained from different measurement devices (e.g., spectrometers made by different manufacturers), potentially improving ML model transferability between them.

It is emphasized that our proposed method shall not be positioned as a replacement for improving study designs and better standardization of classical analytical procedures. Such aspects remain essential when establishing a molecular profiling platform to satisfy i.i.d. assumptions between train-test datasets. In addition to efforts aimed at ensuring train-test dataset comparability, the motivation of our method is to work around the instances when the assumption is violated, due to inevitable sources of error and variability. An inherent limitation of the proposed method is its reliance on a priori knowledge about the sources of possible empirical variations. This presents a challenge as gathering such information may require extensive experimental evaluations of biological and analytical variations. Furthermore, the characterized sources of variability must adequately represent the true possible domain of empirical variability for any successful application. Therefore, continuous refinement through more controlled experiments to include several, independent, sources of variance holds potential to further enhance the utility of the framework. Nevertheless, once the domain of possible variance is successfully characterized for a given system and measurement procedure, the same characterizations may be repeatedly utilized in diverse applications. While our investigations primarily focused on blood-based IR spectroscopy to aid in vitro diagnostics, the practical applications of the CODI framework are not limited to this context. The principle and mathematical foundation of the framework is sufficiently generic to be translated to the examination of diverse biological systems, medical problems, measurement modalities, and ML tasks. Applications involving NMR spectroscopy, mass spectrometry, and Raman spectroscopy serve as direct extensions that can be explored with this modeling framework. Additionally, the framework holds potential in applications related to cell typing and the integration of single-cell multimodal omics data—given the inherent challenges associated with obtaining accurate measurements of cell type populations at scale, where out-of-distribution measurement events are prevalent [60, 61].

Altogether, the presented framework establishes a foundation for future explorations to enhance the robustness of molecular analytics.

### Materials and Methods

More detailed descriptions of the CODI modeling framework, our applications, datasets, experimental procedures, and ML analysis are provided in the Supplementary Information below. The disclosed method according to an optional embodiment which may be described as an in silico model behind CODI may be designed for extensibility, such that it can be applied in various applications and for different measurement modalities. The modeling framework involves utilizing seed observations {s|i=1, . . . , m} that are representative of properties intrinsic to a phenomenon of interest. For instance, a seed observation might represent the mean observation of a class of samples (e.g., healthy control), while another corresponds to a different class (e.g., disease sample). Variability is introduced to the seed observations sᵢ through the addition of random functions f₁, f₂, . . . , fₘ, simulating a measurement as a statistical variable Y in the following generalized form:

Y=sᵢ+f₁+f₂+++fₘ

Repeatedly applying the above model would therefore generate a cohort of simulated measurements, in arbitrary size, centered around sᵢ and incorporating the variations introduced by f₁, f₂, . . . , fₘ. The variations introduced by f₁, f₂, . . . , fₘ can be characterized by ab initio calculations or bottom-up models that each represent a source of expected data variance. However, the former is often specialized and problem specific. An alternative descriptive approach that relies on collecting datasets of calibration measurements which incorporate the levels of expected variance can be easily applied to a variety of problems. For instance, quality control samples can be subjected to different freezer-storage durations, number of freeze/thaw cycles, and aliquoting of samples by different operators. The quality control samples can then be repeatedly measured under different measurement device conditions. The variance observed in this calibration dataset would be reflective of potential sources of variance in handling and measuring samples from the original dataset modeling a certain phenomenon (e.g., biofluids of cases and controls). This variance can then be introduced by defining the function f₁. Other potential sources of data variance, such as biological variability, can be modeled by using additional calibration datasets reflective of the variability sources.

Four independent study cohorts were utilized in our applications of CODI: Lasers4Life-LG [27], BioPersMed [25], KORA [26], and Lasers4Life-Cancer [28]. Lasers4Life-LG involved the collection of blood serum and plasma from 31 healthy individuals initially sampled up to 13 times over a 7-week period, with an additional follow-up after 6 months as detailed in a previous publication [27]. Since this initial publication, the same individuals were invited to participate in two additional sample donations at 3.5 and 4.5 years post their initial involvement. BioPersMed is an ongoing population-based cohort performed at the Medical University Graz, Austria [25]. Repetitive examinations of participants were conducted in 2-year intervals. In the current study, we utilized blood plasma samples and medical data from a subset of 44 healthy individuals (out of 1022 participants). KORA is a population-based cohort in Southern Germany [26]. The cohort comprised of an age- and sex-stratified sample of participants randomly drawn from the resident registration offices within the study area. In the current study, we utilized blood plasma samples and medical data from the second and third visits (named KORA-F4 and KORA-FF4, respectively). The available KORA-F4 data consisted of 3044 samples, while the KORA-FF4 data consisted of 2140 samples. A subset of 2015 individuals participated in both samplings, while 1154 individuals participated in only one of the samplings. Lasers4Life-Cancer is a case-control study cohort involving several cancer entities where both serum and plasma are collected [28]. The samples utilized in this study largely overlapped with samples from our previous study that involved the detection of four common cancer entities (lung, prostate, bladder, and breast) [28]—although the measurement procedures differed (Supplementary Information). Since the original publication, blood plasma and serum samples from different individuals were collected and included in this study. Case samples were collected prior to cancer-related treatment (i.e., therapy-naive). Non-symptomatic controls were pair-matched to cancer cases by age, sex, and body mass index. Experimental measurements of the liquid samples were performed on a Fourier transform IR (FTIR) spectrometer as in previous studies [27, 28, 39]. Samples were injected into a flow cell, and the IR spectra were recorded in transmission mode. The measurement device underwent routine maintenance, with certain components replaced as needed throughout measurements of all clinical samples (Supplementary Information). Multi-class classifications for individual identification were performed using a nearest neighbor algorithm for applications involving training with a single observation per class. For multiclass applications involving training more than one observation per class, a linear discriminant analysis (LDA) algorithm was applied. Binary classifications for case-control applications were performed using a logistic regression algorithm with a ridge penalty. All metrics of classification efficacy were reported on held-out test experimental samples.

### SUPPLEMENTARY INFORMATION

### In Silico Model

In the following subsections, we provide a general framework of the in-silico model behind CODI and the methods according to an optional embodiment of the disclosure to facilitate its extensibility to other applications and molecular fingerprinting modalities. Technical details on our specific applications of the model are provided later in the text.

Generalized Representation CODI may rely on introducing information about possible sources of variations (biological, technical, etc.) that may arise in an experimental setting to an existing set of experimental observations {xᵢ|i=1, . . . , n} where each xᵢ is a numerical vector. In a generalized form, the sources of variability can be denoted as functions f₁, f₂, . . . , fₘ, each representing a model for distinct aspects of variation. These functions are assumed to be random vectors in the same space as the experimental observations, and taking a probability distribution centered around 0.

From the set of experimental observations xᵢ, we can extract another set of experimentally-derived seed observations {s|i=1, . . . , m} that model intrinsic properties of a sub-group in the original set of data—e.g., different classes. With these, a resulting simulated measurement can be modeled as a statistical variable Y, using the following generalized form:

Y=sᵢ+f₁+f₂+ . . . ++fₘ (1)

Repeatedly applying the above model would therefore generate a cohort of simulated measurements, in arbitrary size, centered around sᵢ and incorporating variations introduced by f₁, f₂, . . . , fₘ.

### Setting the Seed Measurements

Defining the experimentally derived seed measurements sᵢ depends on the available input dataset of experimental measurements xᵢ and the type of measurement event we wish to simulate.

In a straightforward implementation, we can set sᵢ=xᵢ. This formulation would allow us to introduce a level of variability to each measurement-specific observation. Repeatedly applying the model for each xxxi would create several sets of measurements, where each set is centered around each experimental observation. In an alternative definition, we can set sᵢ=x̅. This would allow us to introduce a

level of variability to the mean measurement of the experimental set xᵢ, thereby, creating a cohort of measurements centered around their expected value.

If all measurements in xᵢ reflect a certain group or class of samples (e.g., healthy individuals), the simulated cohort as a whole would reflect a measurement distribution of that class of samples. Trivially, when the dataset xᵢ is switched to involve measurements representative of a different class (e.g., disease cases), the simulated cohort would reflect a distribution centered around the alternative outcome.

### Introduction of Variability Modeling Functions

The variations introduced by the functions f₁, f₂, . . . , fₘ may be characterized by ab initio calculations or bottom-up models that each represent a source of expected data variance. However, the former is highly specialized and problem specific. An alternative descriptive approach that relies on collecting datasets of calibration measurements which incorporate the levels of expected variance can be easily applied to a variety of problems.

Suppose an independent calibration dataset {bᵢ|i=1, . . . , l} is available that reflects a given source of measurement variability. Here, f₁ would take the following form:

$${f}_{1} = {\sum }_{i = 1}^{l} β ( 0 , \frac{1}{\sqrt{l}} ) \cdot ( {b}_{i} - \overline{b} ) \qquad ( 2 )$$

By scaling and combining 1 individual deviations from the mean measurement (bᵢ−b̅) using a Gaussian random variable

$$β ( 0 , \frac{1}{\sqrt{l}} )$$

centered around 0, the random function f₁ would have the same variance as the dataset bᵢ. The formal and empirical proof for this was described in a previous study where f₁ generated a level of measurement variance that is observed between different individuals [29]. If the variance source in question does not follow a Gaussian distribution, the random variable β may assume a more appropriate probability distribution.

Additional functions f₂, f₃, . . . , fₘ may be modeled similarly by utilizing different calibration datasets that reflect other sources of measurement variability. In our example applications of the model in this study, the utilized calibration datasets reflected levels of within- or between-person biological variability, along with several sources of analytical variability.

Descriptions on the model variants applied in our applications of the model, along with of how the calibration datasets were defined, is detailed in following sections of the text.

### Simulating Case-Control Measurements

For analysis that involved simulating measurements of cases and controls in our applications (e.g., detection of cancer), the input dataset took the following form, where the superscript denotes a case or control:

$$D = \{ {x}_{1}^{( + 1 )} , {x}_{2}^{( - 1 )} , {x}_{3}^{( - 1 )} , … , {x}_{n}^{( + 1 )} \} \qquad ( 3 )$$

Two model definitions were applied based on Eq. 1, once by setting sᵢ=x̅⁽̅⁺̅¹̅⁾̅ and once by setting sᵢ=x̅⁽̅⁺̅¹̅⁾̅. This results in the following model formulations:

Y⁽⁺¹⁾=x̅⁽̅⁺̅¹̅⁾̅+f₁+f₂+ . . . +fₘY⁽⁻¹⁾=x⁽⁻¹⁾+f₁+f₂+ . . . +fₘ (4)

Both model variants were then repeatedly applied to generate a dataset containing case and control measurements for each disease detection application separately.

### Simulating Longitudinally Captured Measurements

For an application that involved simulating longitudinal measurements of the same individuals, the input dataset took the following form:

$$D = \{ {x}_{0}^{( a )} , {x}_{1}^{( a )} , {x}_{2}^{( a )} , {x}_{0}^{( b )} , … , {x}_{0}^{( z )} , {x}_{1}^{( z )} \} \qquad ( 5 )$$

where the superscript denotes different individuals and the subscript denotes their visit number.

For each individual, we may then utilize their baseline measurement (visit 0) to simulate a set of measurements centered around that baseline in the following form:

$${Y}^{( a )} = {x}_{0}^{( a )} + {f}_{1} + {f}_{2} + … + {f}_{m} \qquad ( 6 )$$

$${Y}^{( b )} = {x}_{0}^{( b )} + {f}_{1} + {f}_{2} + … + {f}_{m}$$

$$⋮$$

$${Y}^{( z )} = {x}_{0}^{( z )} + {f}_{1} + {f}_{2} + … + {f}_{m}$$

Rather than only using the baseline measurements, the same concept can be extended to incorporate additional measurements obtained during subsequent visits for each individual. The process can therefore generate an arbitrary number of measurements for each individual—whether based on a single baseline measurement or more.

### Simulating Between-Person Biological Variability

To model variations in measurements between different individuals, the calibration dataset bᵢ should include experimentally obtained measurements of different individuals. Ideally, all measurements captured for different individuals should be performed in a similar experimental workflow, following the same analytical protocol. This helps ensure that the variance of the calibration dataset is driven

primarily by the biological differences between different individuals, excluding the contributions of other sources of data variance. Here, f₁ may be set to model the between-person biological variability and would be defined in the same way described in Eq. 2.
Simulating within-Person Biological Variability

To model variations in measurements of the same individual over time, the calibration dataset bᵢ should include several experimentally obtained measurements per individual. Similar to the longitudinal dataset definition provided in Eq. 4, we can formulate another calibration dataset of 1 entries that model within-person deviations in the following form:

$$D = \{ {b}_{0}^{a} - \overline{{b}^{a}} , {b}_{1}^{a} - \overline{{b}^{a}} , {b}_{2}^{a} - \overline{{b}^{a}} , … , {b}_{0}^{z} - \overline{{b}^{z}} , {b}_{1}^{z} - {b}^{z} \} \qquad ( 7 )$$

In other terms, the calibration vectors were calculated per individual and the whole set of individual-specific deviations can be pooled into one dataset.

Utilizing the dataset defined in Eq. 7, the function f₁ may be set to model within-person biological variability and would be defined similarly to Eq. 2 with the following form:

$${f}_{1} = {\sum }_{d ϵ D} β ( 0 , \frac{1}{\sqrt{l}} ) \cdot d \qquad ( 8 )$$

With this, the function f₁ thus models the within-person variability as observed across multiple individuals.

### Simulating Analytical Variability

To model variations that may arise from a given analytical procedure, additional experimental measurements that are subjected to experimental variations introduced during the analytical procedure can be utilized. This includes variations that may arise from sample preparation, environmental factors, and instrumental errors. In general, describing the reproducibility of collected data can be achieved by conducting multiple measurements of replicates. Specifically defining how to handle and measure the replicates is dependent on the analytical workflow applied and the measurement technique.

Measurements of replicates can be performed on samples with a high level of technical reproducibility (e.g., water samples) or on quality control samples that exhibit stability in chemical composition similar to that of the intended application (e.g., blood-based samples pooled from thousands of individuals). With blood-based quality control samples, for instance, variations could further include freezer-storage durations, number of freeze/thaw cycles, aliquoting of samples by different operators, varied experimental laboratory room temperature, and sample storage in tubes from different manufacturers.

Technicalities specific to the measurement device may also be intentionally varied when performing measurements—e.g., filter changes, software versions, and cuvette lifetimes. The replicate samples may also be performed across multiple measurement devices, and environmental conditions, including temperature and humidity, to further enrich a calibration dataset.

The variations that arise from the repeated replicate measurements would then be modeled in the same fashion described in Eq. 2. f₂, for instance, may encompass all the sources of measurement variability that are reflected in a blood-based quality control sample, while, f₃ may encompass the sources of measurement variability that are reflected in water measurements.

Modeling such factors, in a way that best mimics what may be encountered in application, makes a given training dataset more resilient to molecular changes or measurement device operating conditions that may arise from an analytical workflow.

Defining which Variability Functions to Include

Successful applications of the CODI modeling framework relies on the careful selection of appropriate sources of variability f₁, f₂, . . . , fₘ. This selection depends on the application of interest and what sources of measurement variability are expected to be observed. For instance, if the envisioned application is to model case-control measurements, it is more critical to include a level of between-person biological variability than to include a level of within-person biological variability. Conversely, if the goal is to simulate how an individual-specific measurement varies over time, including a level of within-person biological variability is necessary.

Sources of analytical errors may be independent of the envisioned application of an analytical procedure (e.g., measurement device drifts, or sample storage variations). Therefore, such variability functions may be included, regardless of the application setting.

In a model validation phase, where experimental test samples are available, it must be ensured that the calibration datasets utilized do not include any of the test samples. This avoids leaking any information or statistical properties of the test samples into the training samples.

In our applications, the variations between quality control samples, between different studies, and between technical replicate measurements were always included in the model. The within-person biological variability was only included for longitudinal applications, while the between-person biological variability was included for case-control applications. The variations between plasma and serum were only included when transferring a model trained on plasma measurements to its application on serum measurements.

Descriptions of what calibration measurements were utilized for each application are provided in the following sections of the text.

### Clinical Studies

### Lasers4Life-LG Study

The Lasers4Life-LG study cohort comprised of 31 nominally healthy individuals, initially sampled up to 13 times over a 7-week period, with an additional follow-up after 6 months as detailed in a previous publication [27]. Since this initial publication, the same individuals were invited to participate in two additional sampling sessions at 3.5 and 4.5 years post their initial involvement. In both the latter two samplings, 8 individuals participated. Blood plasma and serum were collected from all participants throughout the study. The study was approved by the Ethics Committee of the Ludwig-Maximillian-University (LMU) of Munich and all participants provided written informed consent (research study protocol #17-532).

### BioPersMed Study

The BioPersMed study is an ongoing population-based cohort at the Medical University Graz, Austria [25]. Repetitive examinations of participants were conducted in 2-year intervals. In the current study, we utilized blood plasma samples and medical data from a subset of 44 nominally healthy individuals out of 1022 participants. All 44 individuals participated in the baseline sampling, and follow-up visits ranged up to 8 years. Further detailed description of the study design was previously published [25]. The study was approved by the Ethics Committee of the Medical University of Graz, Austria (EC Nr. 24-224 ex 11/12; project application number 4008 22).

### KORA Study

The KORA study is a population-based cohort in Southern Germany [26]. The study comprised of an age- and sex-stratified sample of participants randomly drawn from the resident registration offices within the study area. In the current study, we utilized blood plasma samples and medical data from the second and third participant visits (named KORA-F4 and KORA-FF4, respectively). The available KORA-F4 data consisted of 3044 samples, while the KORA-FF4 data consisted of 2140 samples. A subset of 2015 individuals participated in both samplings, while 1154 individuals participated in only one of the samplings. The samplings were separated by an average of 6.5 follow-up years. Data collection methods and standardized sample collections have been described in detail elsewhere [26, 62-64]. The KORA-F4 and KORA-FF4 study methods were approved by the ethics committee of the Bavarian Chamber of Physicians, Munich (EC No. 06068).

### Lasers4Life-Cancer Study

Samples utilized for the cancer analysis were derived from the case-control Lasers4Life-Cancer study. The samples utilized in this study largely overlap with samples utilized in our previous study that involved the detection of four common cancer entities (lung, prostate, bladder, and breast) [28]. Newly collected blood plasma and serum samples from different individuals were included to increase the sample size and thus improve the statistical robustness of the results. Case samples were collected prior to any cancer-related treatment (i.e., therapy-naive). Non-symptomatic individuals were used as controls. For each cancer entity, controls were pair-matched to the cancer cases by age, sex, and body mass index (BMI). All participants provided written informed consent for the study under research study protocol #17-141 and under research study protocol #17-182, both of which were approved by the Ethics Committee of the LMU of Munich. The clinical trial is registered at the German Clinical Trials Register (ID DRKS00013217).

### EXPERIMENTAL PROCEDURES

### Infrared Spectroscopy

Infrared spectroscopy was performed on liquid samples using Fourier transform IR (FTIR) spectrometer (MIRA Analyzer, CLADE GmbH, Esslingen, Germany). After a sample was injected into a flow cell (window material calcium fluoride) with approx. 8 μm of optical pathlength, the IR spectrum of the sample was recorded in transmission mode and subsequently a reference spectrum of the transport medium (i.e., calcium fluoride saturated water) was recorded. The spectra were acquired with a resolution of 4 cm−1 in a spectral range between 950 cm−1 and 3050 cm−1. The raw data provided by the MIRA Analyzer is the absorption spectrum of the sample subtracted by the reference spectrum. Preprocessing of the resulting IR spectra was performed as described previously [28].

The instrument was maintained by the manufacturer on a yearly basis. The components that were replaced during the yearly or routine user maintenance with potential effect on the IR spectra are the following: light source (after three years); desiccant cartridges (yearly, or when necessary); flow cell when the optical path length exceeded the limit (typically after several thousands of samples), 2 μm flow cell pre-filter (typically after 200-300 samples).

### Sample Handling

All clinical samples involved were stored at −80 °C after being processed to serum or plasma. The transport to measurement laboratory was performed on dry ice, and further storage was at −80 °C. Original samples (0.3 to 1.0 mL) were thawed at 4 °C, and centrifuged for 10 min at 2000 g. The supernatant was aliquoted into the measurement tubes (50-100 μL per tube) and refrozen until measured.

Purchased pooled human serum was used as quality control (QC) samples. 3 liters (100 mL flasks, BioWest, Nuaille, France) were ordered and stored in original flasks at −80 °C. Prior to use, these were thawed at 4 °C, filtered through a 0.45 μm filter, and aliquoted to 50-100 μL aliquots that were kept at −80 °C until use. Each sample measurement batch started and ended with a QC serum measurement.

In addition, a QC sample was measured after every 5 clinical samples. A measurement batch consisted of 25 to 40 samples. Although the same lot of the QC serum was used, slight variability between the flasks were measured, possibly due to the difference in storage duration and technical handling.

To evaluate the technical variability of the FTIR spectrometer, pure water samples were measured. Although not measured daily, several hundreds of water measurements were performed over the years. Due to the pre-processing of measurement data by the FTIR device (i.e., subtraction of the reference spectrum), the resulting deviations reflect the technical noise of the measurement procedure.

### Measurements of Clinical Samples

Samples of the Lasers4Life-LG study were measured in a fully randomized order over all visits but separately for serum and plasma (single flow cell). BioPersMed study samples were measured in a fully randomized order (single flow cell).

### KORA-F4 and KORA-FF4

Study samples were measured in two campaigns separated by an average of 2.7 years between the measurement dates of each sampling. Three flow cells were used throughout both measurement campaigns (KORA-F4: single flow cell; KORA-FF4: two flow cells). For the Lasers4Life-Cancer study, the whole study was split into a training and a test sample set prior to any measurements. Within these sets, the measurement order was fully randomized. The samples of the training set were measured with three flow cells. A gap of four weeks occurred prior to measuring the test set samples. Within the gap, fully independent measurements were routinely performed.

Measurements of the test set were performed on another, fourth, flow cell to simulate potential measurement drifts. All samples were measured in small batches of 25-40 samples plus QC serum as described above. After each measurement batch, the device was cleaned according to the manufacturer's recommendations. A performance qualification test of the instrument was performed daily before the first sample batch or after the clearance of technical issues.

### Simulation and Classification Analysis

### Individual Identification in Longitudinal Monitoring

Simulated datasets of longitudinal measurements were generated using the model formulation presented in Eq. 6. For the analyses illustrated in FIG. 12B-12D, the baseline measurement of each individual served as the initial seed input. In the analysis depicted in FIG. 12E, the model formulation in Eq. 6 was iteratively applied, utilizing up to the first 6 baselines per individual as seed inputs.

Four variability functions were employed to introduce variability to each seed input: (1) within-person biological variability; (2) clinical sampling variability; (3) quality control variability; and (4) technical replicate variability. The within-person variability was always modeled by only using data of two of the three utilized longitudinal cohorts (depicted in FIG. 12A). When the individual identification classification was investigated in one of the three cohorts, the within-person variability was characterized from the two other cohorts. Since the within-person biological variability relied on utilizing follow-up measurements (Eq. 8), this step ensured that no information was leaked to the training sets from the test sets (the follow-ups). For each experimental baseline, 1000 simulated measurements were generated to ensure a sufficiently large sample size, reaching a plateau of classification performance.

Before classification, a data standardization step (mean is 0, standard deviation is 1) and principal component analysis (PCA) were applied. For the data standardization, the mean and standard deviation were calculated only from the training sets to standardize both the training and test sets. PCA was applied to the training sets—keeping components that explain 99.99% of the total variance. The loading vectors from PCA were then applied to both the training sets and test sets before training. After the preprocessing prior to classification, a linear discriminant analysis (LDA) algorithm was used for multi-class classifications when more than one observation per class was available for training. When only one instance per class was available in the training set, a K-nearest neighbor (KNN) algorithm was for classifications (with k=1). Classifier testing was always performed on held-out samples from follow-ups that were not used to create training datasets. Confidence intervals (CIs) were constructed by bootstrap resampling of the test data, following the approach applied in previous work [65]. The [2.5, 97.5] percentile boundaries were chosen to construct a 95% CI interval of the classification accuracy.

Generalization Between Plasma and Serum Specimens For the application that involved creating simulated spectra of plasma and serum mixtures (FIG. 13A), a similar simulation and classification workflow was applied as described in the prior section. However, in addition to the four previously described sources of variability, an additional function was introduced to model deviations between serum and plasma measurements. The calibration datasets used to characterize the differences between IR spectral measurements of blood plasma and serum (depicted in FIG. 13C) were calculated from the Lasers4Life-Cancer study, where plasma and serum were collected at the same sampling occasion. The individual identification application was then applied to the Lasers4Life-LG cohort (FIG. 13F)—an independent cohort that involved different individuals than the Lasers4Life-Cancer cohort.

### Classifier Testing on Independent Test Sets

An L2-regularized logistic regression algorithm was used for the binary (case-control) classification analysis. Data was initially split into a training and test set (FIG. 14A). Three different setups of evaluating the classification efficiency were performed. First, a 10-times repeated 10-fold stratified cross-validation was carried out on the training set of samples. Classification efficiency was evaluated on the validation splits of the cross-validation by calculating the area under the receiver operating characteristic curve (AUC). The AUC was averaged across the validation splits and reported along with its standard deviation. In the second setup, the classifier was trained directly on the experimental data. The AUC was then calculated from the unseen, initially held out, test set. In the third setup, classifier training was performed on a simulated set of samples—based on the training set as seed data. The AUC was again calculated from the unseen, initially held out, experimental test set. When creating the simulated datasets, four variability functions were employed to introduce variability to each seed input: (1) between-person biological variability; (2) clinical sampling variability; (3) quality control variability; and (4) technical replicate variability. The data sources for calculating the level of between-person variability involved the training set of samples, as well as samples from the other independent studies—i.e., samples from all utilized clinical studies, outside of the test set. The model formulation in Eq. 4 was applied, where the mean measurement of cases and controls served as seed measurements for each binary classification task. For each task, 100,000 samples were generated per class.

### Analysis Software

Analysis was performed using custom scripts written in Python (v.3.8.8). The open-source packages NumPy (v.1.21.2), scikit-learn (v.0.24.1), and matplotlib (v.3.5.1) were used.

### 1 Choosing the Variability Functions

The sources of variability that we utilized in our applications of CODI were characterized by sets of calibration measurements that reflect a level of data variance. As previously described, within these calibration measurements were characteristics of empirical variability stemming from inherent biological factors, variations in sample collection and handling, as well as instrument-specific measurement noise and drifts. Specifically, in our longitudinal analyses, we characterized four main sources of variability: (1) within-person biological variability, (2) clinical sampling variability, (3) quality control variability, and (4) technical replicate variability. Here, we examined how each of these sources of variability contributed to the success of the classification. FIG. 15 depicts an analysis where the classification task was to identify an individual given one baseline measurement per individual/class—testing the classification on follow-up measurements. Similar to the analysis depicted in FIGS. 12A and 12B, this analysis was also carried out on the three independent longitudinal cohorts. The CODI modeling framework was employed to generate simulated training data based on all four sources of variability (FIG. 15, left bars). We then systematically removed one of the variability sources considered in the simulation model (keeping the other three) to examine whether the classification accuracy was affected (FIG. 15, remaining bars). We found that with the absence of either the within-person biological variability or the quality control variability, the classification accuracy significantly suffered. In other words, including both of these sources of variability was crucial to the success of the classification across several problems. The variability introduced by the clinical sampling and technical replicate calibration measurements had minimal impact on the classification accuracy. Nevertheless, no significant loss in classification accuracy was observed when all four sources of variability were included in the simulation model. Thus, we included all four sources in our analyses.

FIG. 15 illustrates the impact of the modeled variability sources on the classification accuracy in longitudinal molecular monitoring. Each bar illustrates the classification accuracy achieved by simulating data through CODI with different combinations of the variability sources included.

### 2 Comparative Analysis of Codi to Domain-Agnostic Augmentation Techniques

The CODI modeling framework inherently relies on a priori information on possible sources sample/measurement variability. In contrast, domain-agnostic augmentation methods employ generic transformations (e.g., additive or multiplicative noise) on the input data to simulate new measurements, eliminating the need for a priori information. Here, we provide a comparative analysis between CODI and other domain-agnostic methods of generating simulated training sets. Specifically, for spectral datasets, domain-agnostic augmentation methods often include the introduction of additive white noise, random scaling of intensities by multiplicative factors, linear slope variations, and vertical offset shifts [55, 56](FIG. 16A).

FIG. 16B depicts an analysis where several domain-agnostic augmentation methods were employed to generate simulated training data for the classification task of identifying individuals based on a single baseline measurement per class. As benchmarks, we include the performance of classifiers trained directly on the experimental data and simulated data generated through CODI. Each augmentation

strategy, including CODI, was applied to the same (seed) experimental training data, generating 1000 measurements per class, and tested on the same follow-up experimental measurements. Unlike CODI, the domain-agnostic augmentation methods require the tuning of free parameters that control the extent to which they affect the seed input. Across the three longitudinal cohorts this investigation was carried out, we found that the data generated through CODI had a clear advantage over all other methods.

While the domain-agnostic augmentation techniques are simpler to implement and do not require a priori knowledge, overall they yielded minimal-to-no improvement in the classification accuracy when compared to training directly on the experimental data. Unlike the domain-agnostic augmentation methods, CODI's targeted integration of unrepresented variations allowed for generating more representative simulated data that enhanced the classifier's ability to generalize beyond the original experimental training set.

FIGS. 16A and 16B depict a comparison of CODI to domain-agnostic augmentation schemes. FIG. 16A: Given an input seed experimental spectrum (upper left panel), several methods were employed to generate spectra with added variance. These methods include CODI (bottom left panel), as well as other domain-agnostic augmentation schemes. White noise (upper middle panel) was introduced by repeatedly generating a random Gaussian vector and adding it to the input seed. Multiplicative noise (lower middle panel) was introduced by repeatedly scaling the input seed with random scaling factors. Intensity shifts (upper right panel) were introduced to the input seed by vertically shifting the intensity with random factors. Slope shifts (lower right panel) were introduced by repeatedly manipulating the linear slope of the input seed. FIG. 16B: The individual identification classification task was investigated across three study cohorts. The baseline measurement of each individual in the cohort served as the training seed measurements. Classifiers were trained on the experimental data (left bars), data generated through CODI (second left bars), and data generated through the domain-agnostic augmentation schemes (remaining bars). For each domain-agnostic augmentation scheme, several parameters controlling the data generation were examined, as listed in the legend to the right.

The disclosure refers to the following literature:

### REFERENCES

[1]R. A. Bowen and A. T. Remaley, “Interferences from blood collection tube components on clinical chemistry assays,” Biochemia Medica, pp. 31-44, 2014.
[2]H. Dvinge, R. E. Ries, J. O. Ilagan, D. L. Stirewalt, S. Meshinchi, and R. K. Bradley, “Sample processing obscures cancer-specific alterations in leukemic transcriptomes,” Proceedings of the National Academy of Sciences, vol. 111, p. 16802-16807, November 2014.
[3]P. Yin, R. Lehmann, and G. Xu, “Effects of pre-analytical processes on blood samples used in metabolomics studies,” Analytical and Bioanalytical Chemistry, vol. 407, pp. 4879-4892, March 2015.
[4]S. M. Batool, T. Hsia, A. Beecroft, B. Lewis, E. Ekanayake, Y. Rosenfeld, A. K. Escobedo, A. S. Gamblin, S. Rawal, R. J. Cote, M. Watson, D. T. Wong, A. A. Patel, J. Skog, N. Papadopoulos, C. Bettegowda, C. M. Castro, H. Lee, S. Srivastava, B. S. Carter, and L. Balaj, “Extrinsic and intrinsic preanalytical variables affecting liquid biopsy in cancer,” Cell Reports Medicine, vol. 4, p. 101196, October 2023.
[5]J. Cuklina, C. H. Lee, E. G. Williams, T. Sajic, B. C. Collins, M. R. Martinez, V. S. Sharma, F. Wendt, S. Goetze, G. R. Keele, B. Wollscheid, R. Aebersold, and P. G. A. Pedrioli, “Diagnostics and correction of batch effects in large-scale proteomic studies: a tutorial,” Molecular Systems Biology, vol. 17, August 2021.
[6]C. L. M. Morais, K. M. G. Lima, M. Singh, and F. L. Martin, “Tutorial: multivariate classification for vibrational spectroscopy in biological samples,” Nature Protocols, vol. 15, pp. 2143-2162, June 2020.
[7]J. T. Kwak, R. Reddy, S. Sinha, and R. Bhargava, “Analysis of variance in spectroscopic imaging data from human tissues,” Analytical Chemistry, vol. 84, p. 1063-1069, December 2011.
[8]J. G. Moreno-Torres, T. Raeder, R. Alaiz-Rodriguez, N. V. Chawla, and F. Herrera, “A unifying view on dataset shift in classification,” Pattern Recognition, vol. 45, pp. 521-530, January 2012.
[9]J. P. Cohen, T. Cao, J. D. Viviano, C. W. Huang, M. Fralick, M. Ghassemi, M. Mamdani, R. Greiner, and Y. Bengio, “Problems in the deployment of machine-learned models in health care,” Canadian Medical Association Journal, vol. 193, pp. E1391-E1394, August 2021.
[10]J. R. Zech, M. A. Badgeley, M. Liu, A. B. Costa, J. J. Titano, and E. K. Oermann, “Variable generalization performance of a deep learning model to detect pneumonia in chest radiographs: A cross-sectional study,” PLOS Medicine, vol. 15, p. e1002683, November 2018.
[11]Z. Obermeyer and E. J. Emanuel, “Predicting the future—big data, machine learning, and clinical medicine,” New England Journal of Medicine, vol. 375, pp. 1216-1219, September 2016.
[12]E. Check, “Proteomics and cancer: Running before we can walk?,” Nature, vol. 429, pp. 496-497, June 2004.
[13]M. Peng, Y. Li, B. Wamsley, Y. Wei, and K. Roeder, “Integration and transfer learning of single-cell transcriptomes via cfit,” Proceedings of the National Academy of Sciences, vol. 118, March 2021.
[14]J. A. Gagnon-Bartsch and T. P. Speed, “Using control genes to correct for unwanted variation in microarray data,” Biostatistics, vol. 13, pp. 539-552, November 2011.
[15]R. Molania, J. A. Gagnon-Bartsch, A. Dobrovic, and T. P. Speed, “A new normalization for nanostring nCounter gene expression data,” Nucleic Acids Research, vol. 47, pp. 6073-6083, May 2019.
[16]R. Molania, M. Foroutan, J. A. Gagnon-Bartsch, L. C. Gandolfo, A. Jain, A. Sinha, G. Olshansky, A. Dobrovic, A. T. Papenfuss, and T. P. Speed, “Removing unwanted variation from large-scale RNA sequencing data with PRPS,” Nature Biotechnology, vol. 41, pp. 82-95, September 2022.
[17]A. M. D. Livera, M. Sysi-Aho, L. Jacob, J. A. Gagnon-Bartsch, S. Castillo, J. A. Simpson, and T. P. Speed, “Statistical methods for handling unwanted variation in metabolomics data,” Analytical Chemistry, vol. 87, pp. 3606-3615, March 2015.
[18]J. Liu, Z. Shen, Y. He, X. Zhang, R. Xu, H. Yu, and P. Cui, “Towards out-of-distribution generalization: A survey,” arXiv, 2023.
[19]E. M. Mirkes, J. Bac, A. Fouche, S. V. Stasenko, A. Zinovyev, and A. N. Gorban, “Domain adaptation principal component analysis: Base linear method for learning with out-of-distribution data,” Entropy, vol. 25, p. 33, December 2022.
[20]Y. Chong, Y. Huo, S. Jiang, X. Wang, B. Zhang, T. Liu, X. Chen, T. Han, P. Smith, S.
Wang, and J. Jiang, “Machine learning of spectra-property relationship for imperfect and small chemistry data,” Proceedings of the National Academy of Sciences, vol. 120, May 2023.
[21]X. Zhang, L. Zhou, R. Xu, P. Cui, Z. Shen, and H. Liu, “Towards unsupervised domain generalization,” arXiv, 2022.
[22]X. Li, Y. Dai, Y. Ge, J. Liu, Y. Shan, and L. Duan, “Uncertainty modeling for out-of-distribution generalization,” arXiv, 2022.
[23]A. Mikolajczyk and M. Grochowski, “Data augmentation for improving deep learning in image classification problem,” in 2018 International Interdisciplinary PhD Workshop (IIPhDW), IEEE, May 2018.
[24]C. Shorten, T. M. Khoshgoftaar, and B. Furht, “Text data augmentation for deep learning,” Journal of Big Data, vol. 8, July 2021.
[25]C. W. Haudum, E. Kolesnik, C. Colantonio, I. Mursic, M. Url-Michitsch, A. Tomaschitz, T. Glantschnig, B. Hutz, A. Lind, N. Schweighofer, C. Reiter, K. Ablasser, M. Wallner, N. J. Tripolt, E. Pieske-Kraigher, T. Madl, A. Springer, G. Seidel, A. Wedrich, A. Zirlik, T. Krahn, R. Stauber, B. Pieske, T. R. Pieber, N. Verheyen, B. Obermayer-Pietsch, and A. Schmidt, “Cohort profile: ‘biomarkers of personalised medicine’ (BioPersMed): a single-centre prospective observational cohort study in Graz/Austria to evaluate novel biomarkers in cardiovascular and metabolic diseases,” BMJ Open, vol. 12, p. e058890, April 2022.
[26]R. Holle, M. Happich, H. Lowel, and H. Wichmann, “KORA—a research platform for population based health research,” Das Gesundheitswesen, vol. 67, pp. 19-25, July 2005.
[27]M. Huber, K. V. Kepesidis, L. Voronina, F. Fleischmann, E. Fill, J. Hermann, I. Koch, K. Milger-Kneidinger, T. Kolben, G. B. Schulz, F. Jokisch, J. Behr, N. Harbeck, M. Reiser, C. Stief, F. Krausz, and M. Zigman, “Infrared molecular fingerprinting of blood-based liquid biopsies for the detection of cancer,” eLife, vol. 10, October 2021.
[28]M. Huber, K. V. Kepesidis, L. Voronina, M. Bozic, M. Trubetskov, N. Harbeck, F. Krausz, and M. Zigman, “Stability of” person-specific blood-based infrared molecular fingerprints opens up prospects for health monitoring,” Nature Communications, vol. 12, March 2021.
[29]T. Eissa, K. V. Kepesidis, M. Zigman, and M. Huber, “Limits and prospects of molecular fingerprinting for phenotyping biological systems revealed through in silico modeling,” Analytical Chemistry, vol. 95, no. 16, pp. 6523-6532, 2023.
[30]M. Assfalg, I. Bertini, D. Colangiuli, C. Luchinat, H. Schafer, B. Schutz, and M. Spraul, “Evidence of different metabolic phenotypes in humans,” Proceedings of the National Academy of Sciences, vol. 105, pp. 1420-1424, February 2008.
[31]N. A. Yousri, G. Kastenmuller, C. Gieger, S. Y. Shin, I. Erte, C. Menni, A. Peters, C. Meisinger, R. P. Mohney, T. Illig, J. Adamski, N. Soranzo, T. D. Spector, and K. Suhre, “Long term conservation of human metabolic phenotypes and link to heritability,” Metabolomics, vol. 10, pp. 1005-1017, February 2014.
[32]S. Wallner-Liebmann, L. Tenori, A. Mazzoleni, M. Dieber-Rotheneder, M. Konrad, P. Hofmann, C. Luchinat, P. Turano, and K. Zatloukal, “Individual human metabolic phenotype analyzed by 1H-NMR of saliva samples,” Journal of Proteome Research, vol. 15, pp. 1787-1793, May 2016.
[33]M. Mogri, C. Herzog, J. R. Poganik, J. Justice, D. W. Belsky, A. Higgins-Chen, A. Moskalev, G. Fuellen, A. A. Cohen, I. Bautmans, M. Widschwendter, J. Ding, A. Fleming, J. Mannick, J. D. J. Han, A. Zhavoronkov, N. Barzilai, M. Kaeberlein, S. Cummings, B. K. Kennedy, L. Ferrucci, S. Horvath, E. Verdin, A. B. Maier, M. P. Snyder, V. Sebastiano, and V. N. Gladyshev, “Biomarkers of aging for the identification and evaluation of longevity interventions,” Cell, vol. 186, p. 3758-3775, August 2023.
[34]A. J. Chetwynd, W. B. Dunn, and G. Rodriguez-Blanco, “Collection and preparation of clinical samples for metabolomics,” in Advances in Experimental Medicine and Biology, pp. 19-44, Springer International Publishing, 2017.
[35]M. J. Baker, J. Trevisan, P. Bassan, R. Bhargava, H. J. Butler, K. M. Dorling, P. R. Fielden, S. W. Fogarty, N. J. Fullwood, K. A. Heys, C. Hughes, P. Lasch, P. L. Martin-Hirsch, B. Obinaju, G. D. Sockalingum, J. Sule-Suso, R. J. Strong, M. J. Walsh, B. R. Wood, P. Gardner, and F. L. Martin, “Using fourier transform IR spectroscopy to analyze biological materials,” Nature Protocols, vol. 9, pp. 1771-1791, July 2014.
[36]Z. Yu, G. Kastenmuller, Y. He, P. Belcredi, G. Moller, C. Prehn, J. Mendes, S. Wahl, W. Roemisch-Margl, U. Ceglarek, A. Polonikov, N. Dahmen, H. Prokisch, L. Xie, Y. Li, H. E. Wichmann, A. Peters, F. Kronenberg, K. Suhre, J. Adamski, T. Illig, and R. Wang-Sattler, “Differences between human plasma and serum metabolite profiles,” PLoS ONE, vol. 6, p. e21230, July 2011.
[37]E. Staniszewska, A. K. Bartosz, K. Malek, and M. Baranska, “An effect of anticoagulants on the FTIR spectral profile of mice plasma,” Biomedical Spectroscopy and Imaging, vol. 2, no. 4, p. 317-330, 2013.
[38]C. Soneson, S. Gerster, and M. Delorenzi, “Batch effect confounding leads to strong bias in performance estimates obtained by cross-validation,” PLoS ONE, vol. 9, p. e100335, June 2014.
[39]T. Eissa, C. Leonardo, K. V. Kepesidis, F. Fleischmann, B. Linkohr, D. Meyer, V. Zoka, M. Huber, L. Voronina, L. Richter, A. Peters, and M. Zigman, “Integrative plasma infrared fingerprinting with machine learning enables single-measurement multiphenotype health screening,” Cell Reports Medicine, Manuscript in Review.
[40]R. Gonzalez-Dominguez, A. Gonzalez-Dominguez, A. Sayago, and A. Fernandez-Recamales, “Recommendations and best practices for standardizing the pre-analytical processing of blood and urine samples in metabolomics,” Metabolites, vol. 10, p. 229, June 2020.
[41]J. M. Cameron, H. J. Butler, D. J. Anderson, L. Christie, L. Confield, K. E. Spalding, D. Finlayson, S. Murray, Z. Panni, C. Rinaldi, A. Sala, A. G. Theakstone, and M. J. Baker, “Exploring pre-analytical factors for the optimisation of serum diagnostics: Progressing the clinical utility of ATR-FTIR spectroscopy,” Vibrational Spectroscopy, vol. 109, p. 103092, July 2020.
[42]N. Eling, M. D. Morgan, and J. C. Marioni, “Challenges in measuring and understanding biological noise,” Nature Reviews Genetics, vol. 20, pp. 536-548, May 2019.
[43]C. Lopez-Otin, M. A. Blasco, L. Partridge, M. Serrano, and G. Kroemer, “The hallmarks of aging,” Cell, vol. 153, p. 1194-1217, June 2013.
[44]A. M. Hawkridge and D. C. Muddiman, “Mass spectrometry-based biomarker discovery: Toward a global proteome index of individuality,” Annual Review of Analytical Chemistry, vol. 2, pp. 265-277, July 2009.
[45]C. Lopez-Otin and G. Kroemer, “Hallmarks of health,” Cell, vol. 184, pp. 33-63, January 2021.
[46]S. M. S. F. Rose, K. Contrepois, K. J. Moneghetti, W. Zhou, T. Mishra, S. Mataraso, O. Dagan-Rosenfeld, A. B. Ganz, J. Dunn, D. Hornburg, S. Rego, D. Perelman, S. Ahadi, M. R. Sailani, Y. Zhou, S. R. Leopold, J. Chen, M. Ashland, J. W. Christle, M. Avina, P. Limcaoco, C. Ruiz, M. Tan, A. J. Butte, G. M. Weinstock, G. M. Slavich, E. Sodergren, T. L. McLaughlin, F. Haddad, and M. P. Snyder, “A longitudinal big data approach for precision health,” Nature Medicine, vol. 25, pp. 792-804, May 2019.
[47]S. Guo, J. Popp, and T. Bocklitz, “Chemometric analysis in raman spectroscopy from experimental design to machine learning-based modeling,” Nature Protocols, vol. 16, pp. 5426-5459, November 2021.
[48]L. Haghverdi, A. T. L. Lun, M. D. Morgan, and J. C. Marioni, “Batch effects in single-cell RNA-sequencing data are corrected by matching mutual nearest neighbors,” Nature Biotechnology, vol. 36, pp. 421-427, April 2018.
[49]R. A. Zanini and E. L. Colombini, “Parkinson's disease EMG data augmentation and simulation with DCGANs and style transfer,” Sensors, vol. 20, p. 2605, May 2020.
[50]F. Wang, S. Zhong, J. Peng, J. Jiang, and Y. Liu, “Data augmentation for EEG-based emotion recognition with deep convolutional neural networks,” in MultiMedia Modeling, pp. 82-93, Springer International Publishing, 2018.
[51]F. Lotte, “Signal processing approaches to minimize or suppress calibration time in oscillatory activity-based brain-computer interfaces,” Proceedings of the IEEE, vol. 103, pp. 871-890, June 2015.
[52]D. Freer and G. Z. Yang, “Data augmentation for self-paced motor imagery classification with c-LSTM,” Journal of Neural Engineering, vol. 17, p. 016041, January 2020.
[53]P. Tsinganos, B. Cornelis, J. Cornelis, B. Jansen, and A. Skodras, “Data augmentation of surface electromyography for hand gesture recognition,” Sensors, vol. 20, p. 4892, August 2020.
[54]S. Guo, R. Heinke, S. Stockel, P. Rosch, J. Popp, and T. Bocklitz, “Model transfer for Raman-spectroscopy-based bacterial classification,” Journal of Raman Spectroscopy, vol. 49, no. 4, pp. 627-637, 2018.
[55]E. J. Bjerrum, M. Glahder, and T. Skov, “Data augmentation of spectral data for convolutional neural network (CNN) based deep chemometrics,” arXiv, 2017.
[56]A. Lebrun, H. Fortin, N. Fontaine, D. Fillion, O. Barbier, and D. Boudreau, “Pushing the limits of surface-enhanced raman spectroscopy (SERS) with deep learning: Identification of multiple species with closely related molecular structures,” Applied Spectroscopy, vol. 76, p. 609-619, March 2022.
[57]K. Sohn, D. Berthelot, C. L. Li, Z. Zhang, N. Carlini, E. D. Cubuk, A. Kurakin, H. Zhang, and C. Raffel, “Fixmatch: Simplifying semi-supervised learning with consistency and confidence,” arXiv, 2020.
[58]E. Bareinboim and J. Pearl, “Causal inference and the data-fusion problem,” Proceedings of the National Academy of Sciences, vol. 113, p. 7345-7352, July 2016.
[59]T. Kyono and M. van der Schaar, “Exploiting causal structure for robust model selection in unsupervised domain adaptation,” IEEE Transactions on Artificial Intelligence, vol. 2, p. 494-507, December 2021.
[60]S. Dorkenwald, P. H. Li, M. Januszewski, D. R. Berger, J. Maitin-Shepard, A. L. Bodor, F. Collman, C. M. Schneider-Mizell, N. M. da Costa, J. W. Lichtman, and V. Jain, “Multi-layered maps of neuropil with segmentation-guided contrastive learning,” Nature Methods, vol. 20, p. 2011-2020, November 2023.
[61]R. Argelaguet, A. S. E. Cuomo, O. Stegle, and J. C. Marioni, “Computational principles and challenges in single-cell data integration,” Nature Biotechnology, vol. 39, p. 1202-1215, May 2021.
[62]H. E. Wichmann, C. Gieger, and T. Illig, “KORA-gen—resource for population genetics, controls and a broad spectrum of disease phenotypes,” Das Gesundheitswesen, vol. 67, pp. 26-30, July 2005.
[63]C. Sujana, J. Seissler, J. Jordan, W. Rathmann, W. Koenig, M. Roden, U. Mansmann, C. Herder, A. Peters, B. Thorand, and C. Then, “Associations of cardiac stress biomarkers with incident type 2 diabetes and changes in glucose metabolism: KORA F4/FF4 study,” Cardiovascular Diabetology, vol. 19, October 2020.
[64]W. Rathmann, B. Haastert, A. Icks, H. Lowel, C. Meisinger, R. Holle, and G. Giani, “High prevalence of undiagnosed diabetes mellitus in southern germany: Target populations for efficient screening. the KORA survey 2000,” Diabetologia, vol. 46, pp. 182-189, February 2003.
[65]B. Sanchez-Lengeling, J. N. Wei, B. K. Lee, R. C. Gerkin, A. Aspuru-Guzik, and A. B. Wiltschko, “Machine learning for scent: Learning generalizable perceptual representations of small molecules,” arXiv, 2019.

### LIST OF REFERENCE CHARACTERS

100 method for analyzing experimental data
102-108 method steps
120 data processing device
200 method of generating training data for training a machine learning model for analyzing experimental data
202-208 method steps
300 computer-implemented method for training a machine learning model for analyzing experimental data
302-304 method steps
400 computer-implemented method for analyzing experimental data by using a machine learning model
500 computer implemented method of analyzing experimental data by using a machine learning model
600 data processing device
700 computer-implemented method for classifying experimental data being subject to variation
800 method of identifying an individual based on a specimen of a biofluid of the individual
802-806
900 computer-implemented method of generating data for analyzing experimental data

What is claimed is:

1. A computer-implemented method for analyzing experimental data, the method comprising:
receiving a first data set including the experimental data to be analyzed;
receiving one or more variation data sets, wherein each variation data set includes information regarding a variation of experimental observations;
extrapolating the experimental data by applying a variation to the experimental data, wherein the applied variation corresponds to the one or more variations of the experimental observations specified by the information comprised in the one or more variation data sets;
generating a synthetic data set based on the extrapolated experimental data;
analyzing the synthetic data set; and
providing an output based on a result of the analysis of the synthetic data set;
wherein the variation affecting the experimental data to be analyzed originates at least partially in a variation between types of samples from which the experimental data is extracted; and in that the one or more variation data sets are selected depending on one or more types of noise related to the experimental data to be analyzed.

2. The method according to claim 1, wherein the one or more variation data sets are selected such that the variation of experimental observations included in the one or more data sets correspond to noise typically affecting experimental data of a same type as the experimental data included in the first data set.

3. The method according to claim 1, wherein the one or more variation data sets are selected in a contextual manner based on at least one of a context related to the experimental data to be analyzed and based on a context related to the analysis of the experimental data to be performed.

4. The method according to claim 1, wherein the one or more variation data sets are at least partially selected and/or provided by an external source.

5. The method according to claim 1, further comprising following steps prior to receiving the one or more variation data sets:
determining one or more types of noise to be considered when analyzing the experimental data; and
selecting the one or more variation data sets based on the determined one or more types of noise.

6. The method according to claim 1, wherein the experimental observations of the one or more variation data sets are independent of the experimental data included in the first data set.

7. The method according to claim 1, wherein extrapolating the experimental data comprises or consist of adding a measurement point cloud for at least one measurement point of the experimental data, wherein the measurement point cloud is based on at least one of the one or more variation data sets.

8. The method according to claim 1, wherein the one or more variation data sets may be selected such that the experimental observations of the one or more variation data sets and the experimental data included in the first data set are of a same type of experimental data.

9. The method according to claim 8, wherein at least one of following is true:
a type of the experimental data relates to spectroscopic data;
the experimental data includes data retrieved by at least one of following techniques:
vibrational spectroscopy; optical spectroscopy in a visible and/or ultraviolet spectral range; X-ray diffraction; NMR spectroscopy; mass spectrometry; clinical chemistry tests; electrophysiology; and flow cytometry;
the experimental data is retrieved from a biofluid of a biological organism; and
the biofluid includes at least one of the following types: blood; plasma; serum; saliva; urine or exprimate urine; cerebrospinal fluid; living or chemically fixed animal, human, plant tissue; living or chemically fixed bacterial, animal, human, plant cells; viruses or viral particles; and multimolecular structures.

10. The method according to claim 1, wherein the variation of the experimental observations comprised in a particular variation data set of the one or more variation data sets originates in one of following sources of variation:
a tolerance in quantitative measurement reproducibility of a measurement technique used for retrieving the experimental observations;
a preanalytical variability occurring in a process for preparing one or more samples for retrieving the experimental observations;
a variation between different phenotypes from which the one or more samples for retrieving the experimental observations originate; and
a variation that is inherent to the studied biological system, encompassing both variations between different individuals and intra-individual variations when studied over time.

11. A non-transitory computer-readable storage medium, comprising instructions which, when executed by a computer, cause the computer to carry out the method according to claim 1.

12. A data processing device, comprising a processor and a memory, wherein the memory stores instructions which, when executed by the processor, cause the data processing device to carry out the method according to claim 1.

13. A computer-implemented method of generating training data for training a machine learning model for analyzing experimental data, the method comprising:
receiving one or more variation data sets, wherein each variation data set includes experimental observations being subject to a variation at least partially affecting the experimental data to be analyzed, wherein the variation affecting the experimental data to be analyzed originates at least partially in a variation between types of samples from which the experimental data is extracted;
analyzing the variation of the experimental observations comprised in the one or more variation data sets and determining variability properties based on the analyzed variation;
receiving a first data set including at least one reference specimen of the experimental data and a predetermined analysis result for the at least one reference specimen; and
generating one or more synthetic data sets including multiple synthetic data points by applying the determined variability properties to the first data set.

14. The method according to claim 13, wherein a number of synthetic data points is larger than the number of the at least one reference specimen of the experimental data included in the first data set.

15. The method according to claim 13, wherein the one or more variation data sets are selected, such that the variation of the experimental observations included in the one or more variation data sets corresponds to an actual variation and/or an expected variation affecting the experimental data to be analyzed.

16. The method according to claim 13, wherein the variation affecting the experimental data to be analyzed originates in one or more of following variation sources:
a tolerance in quantitative measurement reproducibility of a measurement technique used for retrieving the experimental data;
a preanalytical variability occurring in a process for preparing one or more samples for retrieving the experimental data;
a variation between different phenotypes from which the one or more samples for retrieving the experimental data originate; and
a variation that is inherent to the studied biological system, encompassing both variations between different individuals and intra-individual variations when studied over time.

17. The method according to claim 16, wherein each variation data set includes at least one of experimental observations affected by a variation originating in one or more predetermined variation sources, and experimental observations affected solely by a variation originating in one or more predetermined sources of the variation sources.

18. The method according to claim 16, wherein each variation data set includes experimental observations affected by a variation originating in one or more predetermined variation sources, wherein the experimental observations are essentially unaffected by variations originating in variation sources different from the predetermined variation sources.

19. The method according to claim 13, wherein the reference specimen of the experimental data included in the first data set is retrieved from a same and/or a different biofluid type as the experimental data to be analyzed by the machine learning model to be trained with the one or more synthetic data sets.

20. The method according to claim 13, wherein the one or more variation data sets are selected such that the experimental observations included in the one or more variation data sets are affected by a same and/or a similar type of variation as the experimental data to be analyzed.

21. The method according to claim 13, wherein analyzing the variation of the experimental observations comprised in the one or more variation data sets includes analyzing the variation of the experimental observations of each of the one or more variation data sets independently of each other; and/or wherein determining the variability properties based on the analyzed variation of the experimental observations includes determining the variability properties based on the experimental observations for each of the one or more variation data sets independently of each other.

22. The method according to claim 13, wherein applying the determined variability properties to the first data set comprises extrapolating each of at least some of the one or more data points of the reference specimen of the experimental data included in the first data to multiple synthetic data points according to the variability properties.

23. The method according to claim 13, wherein the synthetic data set is provided as training data for training a machine learning model for analyzing experimental data based on out-of-distribution generalization.

24. A computer-implemented method for training a machine learning model for analyzing experimental data, the method comprising:
receiving a training data set including training data, wherein the training data includes one or more synthetic data sets having a same or a similar variation as one or more variation data sets used for generating the one or more synthetic data sets based on a first data set including at least one reference specimen of the experimental data and a predetermined analysis result for the at least one reference specimen; and
training the machine learning model with the received training data set.

25. Computer-implemented method for analyzing experimental data by using the machine learning model trained with the method according to claim 24, wherein the machine learning model is configured to classify the analyzed experimental data being subject to a variation exceeding a variation of reference specimen of the experimental data used as a first data set for generating the training data for training the machine learning model.

26. A non-transitory computer-readable storage medium, comprising instructions which, when executed by a computer, cause the computer to carry out the method according to claim 24.

27. A data processing device, comprising a processor and a memory, wherein the memory stores instructions which, when executed by the processor, cause the data processing device to carry out the method according to claim 24.
